SEQUENCE LISTING (2 sequences)

SEQ ID NO: 1 - protein, 16 aa, synthetic construct
ATWLPPRAANLLMAAS

SEQ ID NO: 2 - protein, 12 aa, synthetic construct
FPNWSLRPMNQM

(19) United States
(12) Patent Application Publication
(10) Pub. No.: US 2025/0177423 A1
(43) Pub. Date: Jun. 5, 2025

(54) PHOTORESPONSIVE PEPTIDE DELIVERY SYSTEM AND METHOD OF USING THE SAME

(71) Applicants: THE UNIVERSITY OF HONG KONG, Hong Kong (CN); Zhongshan Ophthalmic Center, Sun Yat-sen University, Guang Dong (CN)

(72) Inventors: Weiping WANG, Hong Kong (CN); Xiaoling LIANG, Hong Kong (CN); Yang ZHOU, Guang Dong (CN); Jia LI, Hong Kong (CN)

(73) Assignees: THE UNIVERSITY OF HONG KONG, Hong Kong (CN); Zhongshan Ophthalmic Center, Sun Yat-sen University, Guang Dong (CN)

(21) Appl. No.: 18/966,507

(22) Filed: Dec. 3, 2024

(30) Foreign Application Priority Data
Dec. 4, 2023 (CN) 202311653291.X

(51) Int. Cl. A61K 31/69; A61K 9/00; A61K 41/00; A61K 45/06; A61K 47/64; A61K 47/69; A61P 7/00; C07K 7/08

(52) U.S. Cl. CPC A61K 31/69; A61K 9/0019; A61K 41/0042; A61K 45/06; A61K 47/64; A61K 47/6935; A61P 7/00; C07K 7/08

(57) ABSTRACT

Provided herein is a photoresponsive prodrug comprising an active agent conjugated to a photoresponsive group, and a nanoparticle, wherein the photoresponsive prodrug is co-assembled with a polymer to form a nanoparticle. Also provided is a method of treating a subject, comprising administering photoresponsive prodrug or the nanoparticle to the subject, and irradiating at the target site with a light source.

### RELATED APPLICATIONS

This application claims priority to Chinese patent application No. 202311653291.X, filed Dec. 4, 2023 which is incorporated by reference in its entirety.

### 1. FIELD

This invention is generally in the field of synthetic prodrugs, nanoparticle and their use in treating or preventing diseases.

### SEQUENCE LISTING

A sequence listing, prepared on Nov. 20, 2024 as the ST26 text file “F23P4978-sequence listing.xml” having file size of 2621 bytes, is incorporated by reference in its entirety.

### 2. BACKGROUND

In recent decades, diseases of the posterior eye segment such as age-related macular degeneration, diabetic retinopathy, and posterior uveitis have drawn much attention since the damage to the retina and optic nerve is the main cause of sever vision loss. Due to the unique anatomy and physiology of the eyes, the delivery of adequate doses of drugs to the posterior segment of the eyes remains challenging and systemic administration of high dose drugs usually cause severe systemic adverse events. Although intravitreal injections have been proven to be a good solution, the local adverse events by repeating surgical procedure including cataract, endophthalmitis, vitreous hemorrhage are sometimes observed, let alone the mental stress to some patients.

The photoresponsive nanocarriers for targeted and sustainable delivery of drugs to the posterior segment of eyes have been extensively investigated in recent years, since the light can easily reach the posterior segment of the eyes through the tissues in front including cornea, lens, and vitreous body, which are usually transparent in nature. Although it is widely recognized that the short wavelength light can induce damage to multiple eye tissues especially the retina due to phototoxicity, most nanocarriers reported still requires the short-wavelength light for triggering the drug release, since complex fabrication processes or toxic components will be needed otherwise and two-photon excitation mechanism or photon upconversion process typically requires pulsed lasers with high power sources.

So far, there remains a need for photoresponsive prodrug delivery systems with improved therapeutic efficacy, better circulation stability and less phototoxicity.

### 3. SUMMARY

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of features.

In one aspect, the present invention relates to a photoresponsive prodrug comprising an active agent conjugated to a photoresponsive group. Such a prodrug is useful in achieving light-controlled delivery of peptides as well as the treatment and prevention of various diseases.

In some embodiments, the active agent is an anti-angiogenic agent. In some embodiments, the active agent is an anti-angiogenic peptide. In some embodiments, the active agent is AS16 peptide with a sequence of ATWLPPRAANLLMAAS (SEQ ID NO: 1) or FM12 peptide comprising an amino acid sequence of FPNWSLRPMNQM (SEQ ID NO: 2).

In some embodiments, the photoresponsive group is boron-dipyrromethene (BODIPY). In some embodiments, the BODIPY is a moiety selected from formula (VII), formula (VIII), formula (IX), formula (X), formula (XI), or formula (XII). In some embodiments, the BODIPY is a moiety of formula (XII):

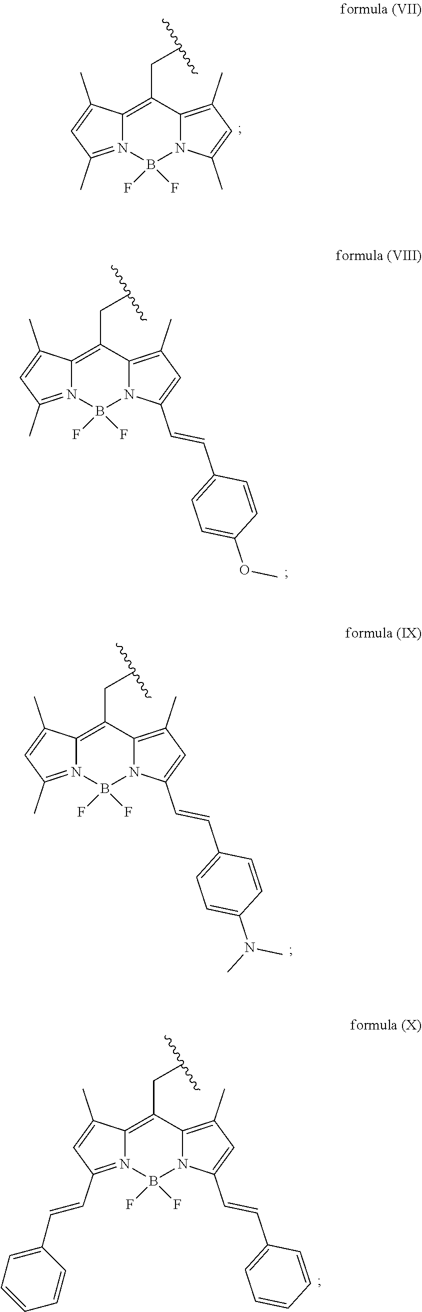

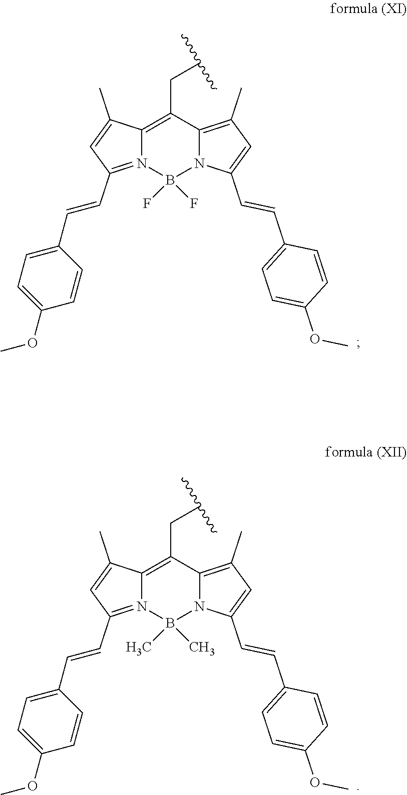

In some embodiments, the photoresponsive prodrug is:

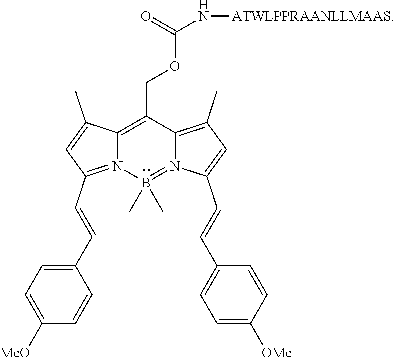

In another aspect, provided herein is a nanoparticle, comprising the photoresponsive prodrug as disclosed herein, and one or more polymers.

In some embodiments, the polymer is selected from polylactic acid (PLA), polyethylene glycol (PEG), polydimethylsiloxane (PDMS), polyethyleneimine (PEI), polyamidoamine (PAMAM), and a combination thereof. In some embodiments, the polymer is PLA-PEG copolymer. In some embodiments, the polymer is PLA5k-PEG2k or PEG3.4k-PLA5k.

In some embodiments, the nanoparticle has a diameter of about 20-200 nm. In some embodiments, the nanoparticle has a diameter of about 40-100 nm. In some embodiments, the nanoparticle has a diameter of about 50-70 nm.

In some embodiments, the nanoparticle has a polydispersity index (PDI) of 0.120-0.220. In some embodiments, the nanoparticle has a polydispersity index (PDI) of 0.150-0.200.

In some embodiments, the nanoparticle has a diameter of about 60 nm, and a PDI of 0.170; or has a diameter of about 122 nm, and a PDI of 0.2.

In some embodiments, the nanoparticle comprises a photoresponsive prodrug of the following formula co-assembled with PLA5k-PEG2k

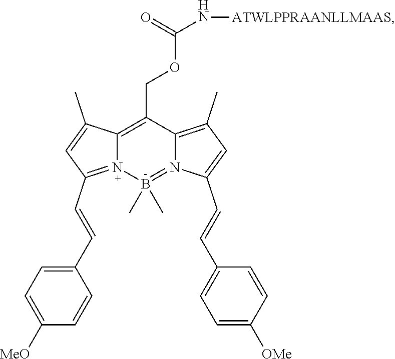

or a photoresponsive prodrug of cFM12 co-assembled with PEG3.4k-PLA5k.

In some embodiments, the nanoparticle further encapsulates additional agents. In some embodiments, the additional agents are hydrophobic drugs. In some embodiments, the additional agents are anti-cancer drugs. In some embodiments, the additional agents are selected from tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfm, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, or iniparib. In some embodiments, the additional agent is elesclomol.

In another aspect, provided herein is a pharmaceutical composition, comprising: (i) the photoresponsive prodrug as disclosed herein or the nanoparticle as disclosed herein; and (ii) a pharmaceutically acceptable excipient.

In yet another aspect, provided herein is a method of delivering a drug to a subject at a target site, comprising: (i) administering the photoresponsive prodrug as disclosed herein, the nanoparticle as disclosed herein or the pharmaceutical composition disclosed herein to the subject; and (ii) irradiating the self-assembly system at the target site with a light source.

In some embodiments, the light source has a wavelength of about 500-1300 nm. In some embodiments, the light source has a wavelength of about 600-900 nm. In some embodiments, the light source has a wavelength of about 600-700 nm. In some embodiments, the light source has a wavelength of about 656 nm.

In some embodiments, the photoresponsive drug or the nanoparticle as disclosed herein is administered via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, intratumoral administration, and any combination thereof. In some embodiments, the photoresponsive drug or the nanoparticle as disclosed herein is administered via intravenous administration.

In some embodiments, the target site is eye, skin, or tumor. In some embodiments, the target site is posterior segment of the eye.

In another aspect, provided herein is a method for treating or preventing a disease characterized by abnormal angiogenesis, comprising: (i) administering the photoresponsive prodrug as disclosed herein, the nanoparticle as disclosed herein or the pharmaceutical composition disclosed herein to the subject; and (ii) irradiating the self-assembly system at the target site with a light source.

In some embodiments, the light source has a wavelength of about 500-1300 nm. In some embodiments, the light source has a wavelength of about 600-900 nm. In some embodiments, the light source has a wavelength of about 600-700 nm. In some embodiments, the light source has a wavelength of about 656 nm.

In some embodiments, the photoresponsive drug or the nanoparticle as disclosed herein is administered via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, intratumoral administration, and any combination thereof. In some embodiments, the photoresponsive drug or the nanoparticle as disclosed herein is administered via intravenous administration.

In some embodiments, the target site is eye, skin, or tumor. In some embodiments, the target site is posterior segment of the eye.

In some embodiments, the method is for the treatment or prevention of cancer. In some embodiments, the method is for the treatment or prevention of kidney cancer, lung cancer, breast cancer, colon cancer, prostate cancer, brain cancer, chondrosarcoma or angiosarcoma.

In some embodiments, the method is for the treatment or prevention of ocular neovascular disease. In some embodiments, the method is for the treatment or prevention of age-related macular degeneration (AMD), choroidal neovascularization secondary to myopia, proliferative diabetic retinopathy, diabetic macular edema, retinal vascular occlusions such as retinal vein occlusion, ocular tumors, Hipper-Lindau syndrome, retinopathy of prematurity, and polypoid choroidal vasculopathy. In some embodiments, the method is for the treatment or prevention of AMD, especially exudative AMD (wet AMD).

In another aspect, provided herein is a kit, comprising the photoresponsive prodrug as disclosed herein, the nanoparticle as disclosed herein, or the pharmaceutical composition as disclosed herein.

### 4. BRIEF DESCRIPTION OF DRAWINGS

### 4. Brief Description of the Figures

The patent or application file contains at least one drawing executed in color. Copies of this patent or patent application publication with color drawings will be provided by the Office upon request and payment of the necessary fee.

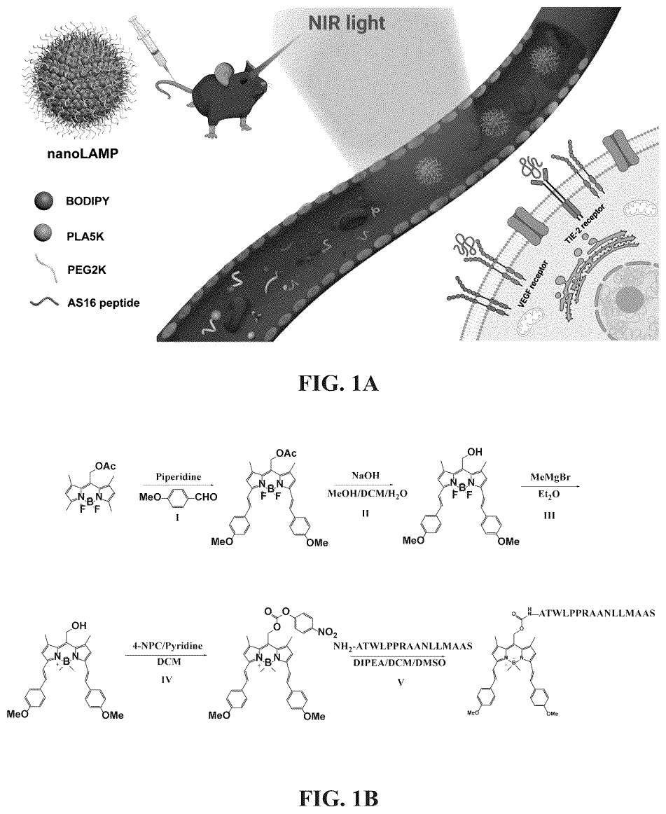

FIGS. 1A-B. Schematic illustration of the NIR light responsive AS16 peptide release nanosystem for the treatment of choroidal neovascularization. (A) 3D structure of nanoLAMPs composed of BODIPY-AS16 conjugates and PLA5k-PEG2k polymers and illustration of function. NIR light triggers the release of AS16 at desired spot for targeted anti-angiogenesis and anti-M2 macrophage polarization therapy. (B) Synthesize route of the BODIPY-AS16 conjugate.

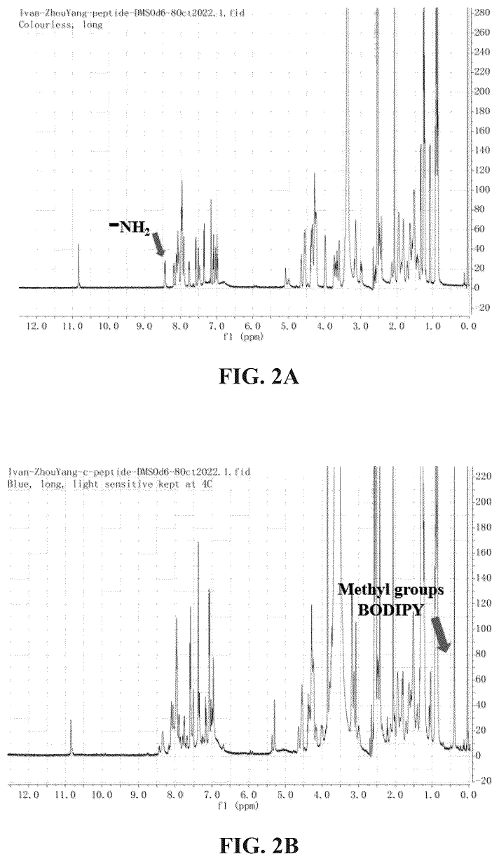

FIGS. 2A-B. ¹H-NMR spectrum uncaged (A) and BODIPY-caged AS16 peptide (B).

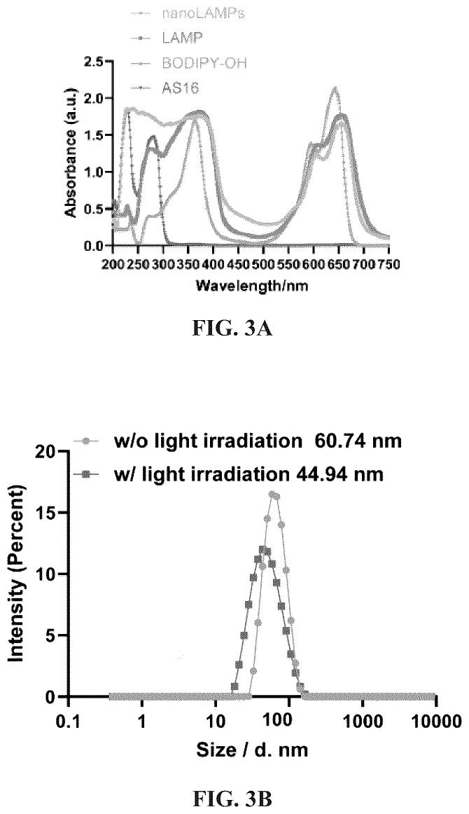

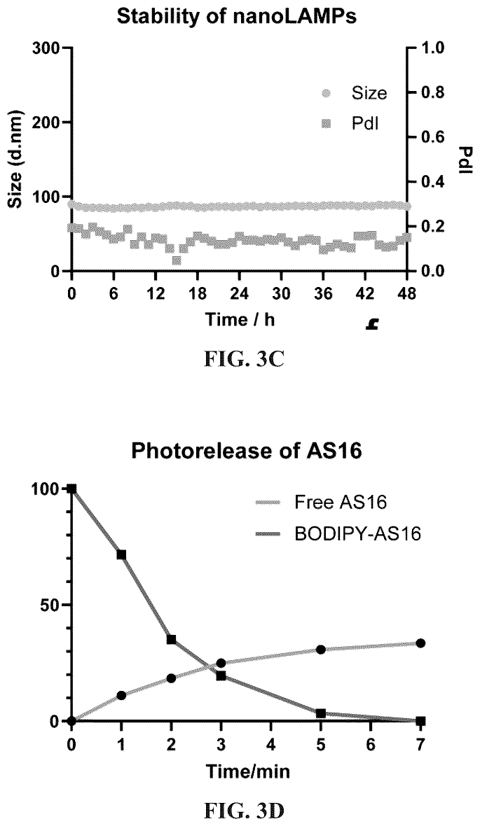

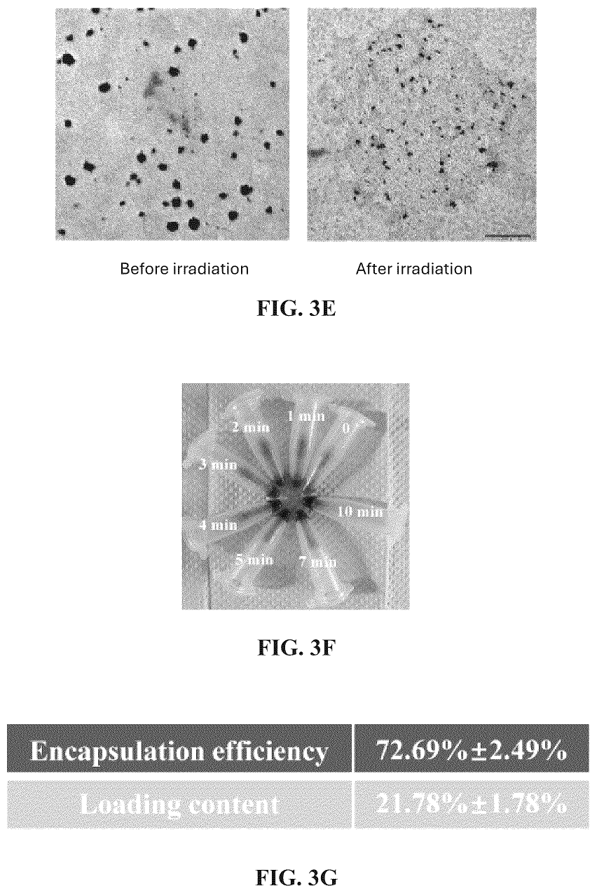

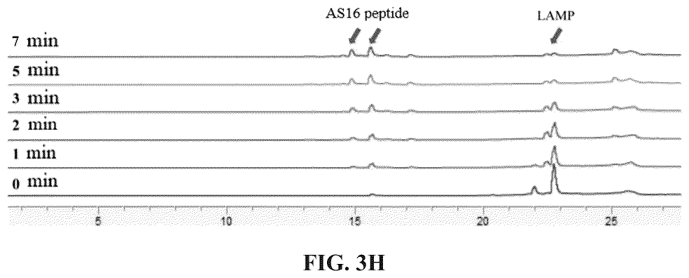

FIGS. 3A-H. Characteristics of nanoLAMPs. (A) UV-Vis spectra of nanoLAMPs, LAMPs, BODIPY-OH, and AS16 peptides (B) Dynamic size change of nanoLAMPs after NIR light irradiation revealed by DLS. (C) Results of the stability test of nanoLAMPs in DMEM complete medium at 37° C. by DLS. (D) Quantified photo release profile of AS16 peptide from HPLC analysis. (E) TEM images of nanoLAMPs before and after NIR light irradiation. (F) Color change of nanoLAMPs after NIR light irradiation for different time period. (G) Loading capacity and encapsulation efficiency of BODIPY-AS16 conjugates in nanoLAMPs. (H) HPLC spectra showing the release of AS16 peptide. The data are presented as mean±SEM.

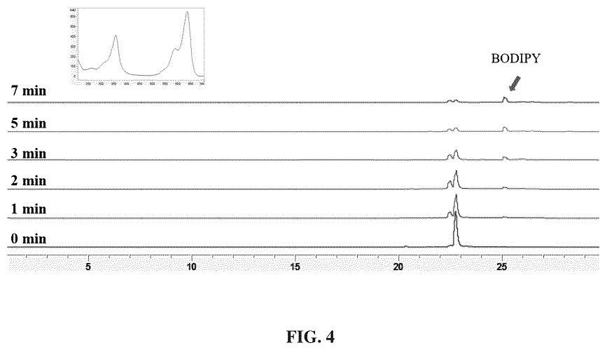

FIG. 4. HPLC spectra of LAMP under NIR light irradiation for different time periods, indicating the release of BODIPY from LAMP following NIR light irradiation.

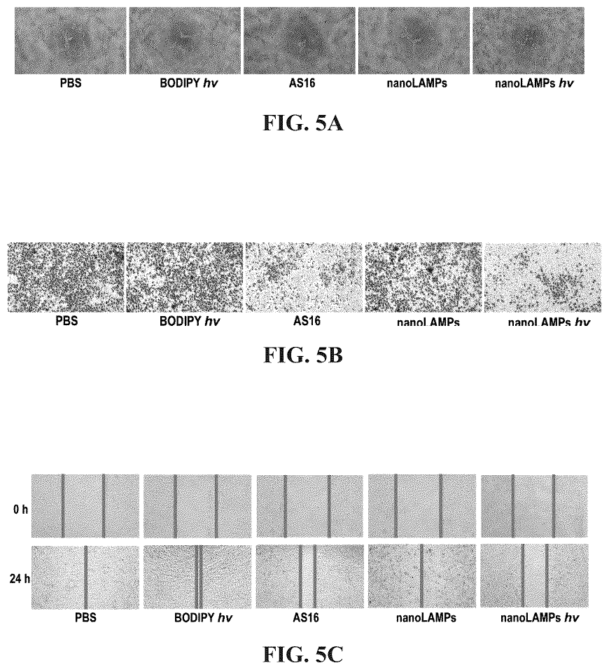

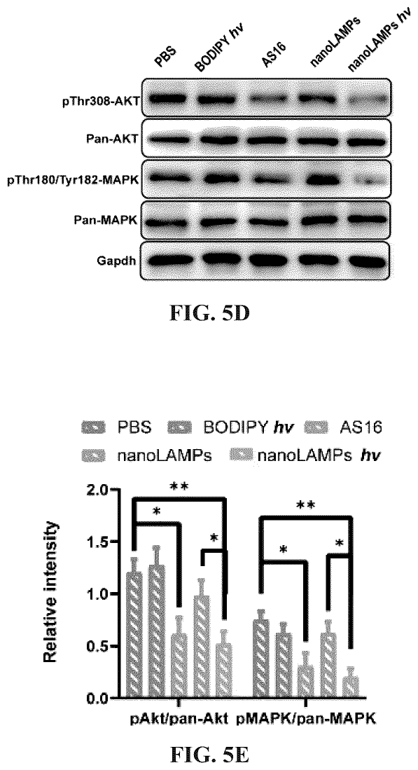

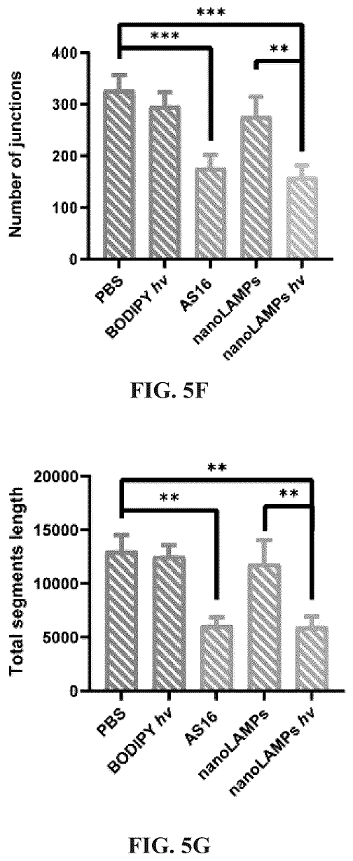

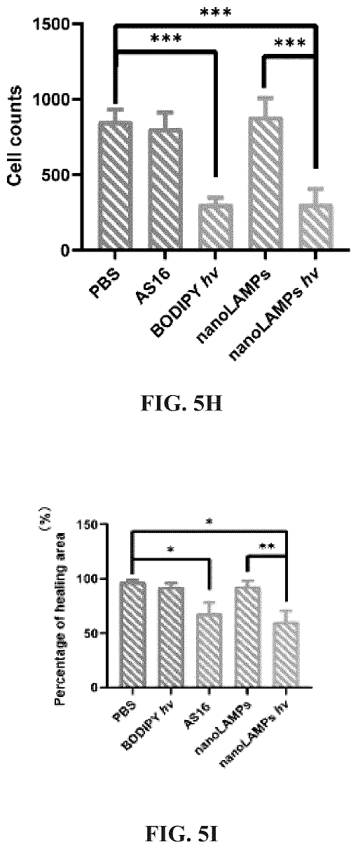

FIGS. 5A-I. In vitro NIR induced AS16 peptide release from nanoLAMPs for anti-angiogenesis therapy. (A) VEGF stimulated HUVECs tube formation. HUVEC suspensions were treated with 20 ng/mL VEGF165 and co-incubated with different formulations at a AS16 peptide equivalent dose of 5 μM. nanoLAMPs with light irradiation group showed significantly less tube structure formation compared with the control group and nanoLAMPs without light irradiation group (n=3). (B) Migration assay of each group at 24 h. the trend was similar with the tube formation assay (n=3). (C) Representative images of wound healing assay of each group, which showed superior therapeutic efficacy in nanoLAMPs with light irradiation group (n=3). (D) Representative images of Western blot analysis of pro-angiogenic signaling proteins in HUVECs after different treatment (n=3). (E) Statistical analysis of western blot analysis as calculated by ImageJ. (F) Statistical analysis of number of junctions in tube formation assay. (G) Statistical analysis of total segment length of each group. (H) Statistical analysis of cell counts on the bottom chamber of each group. (I) Statistical analysis of wound healing process of each group measured by ImageJ software. (*: p<0.05; **: p<0.01; ***: p<0.001). The data are presented as mean±SEM.

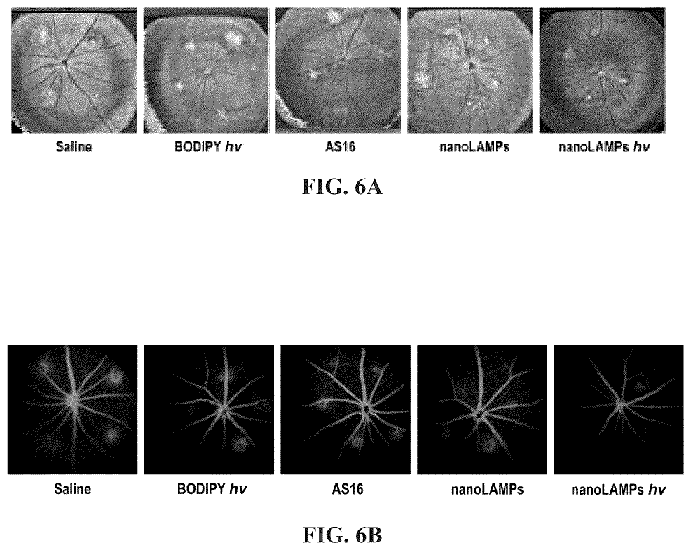

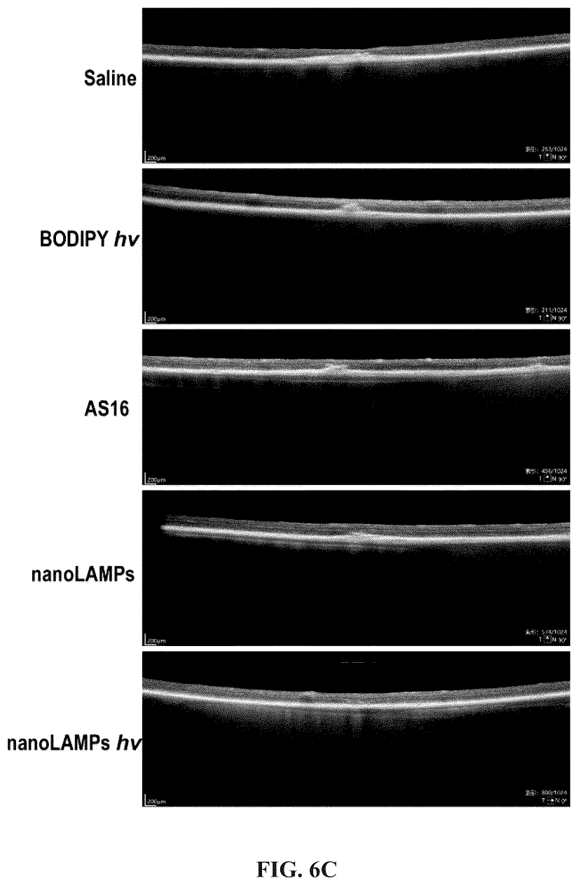

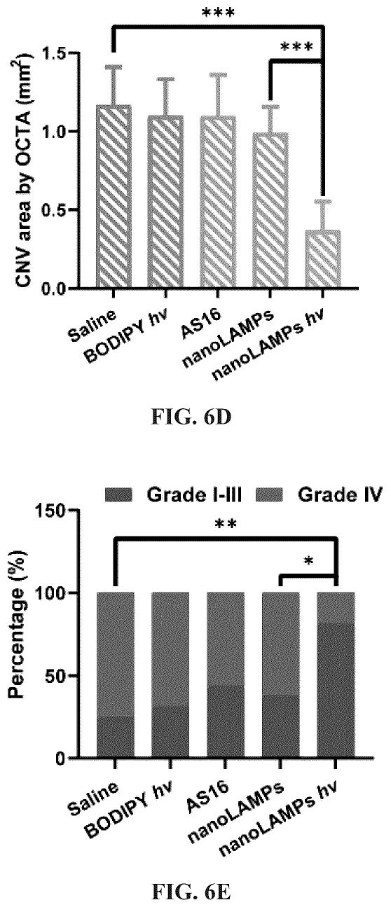

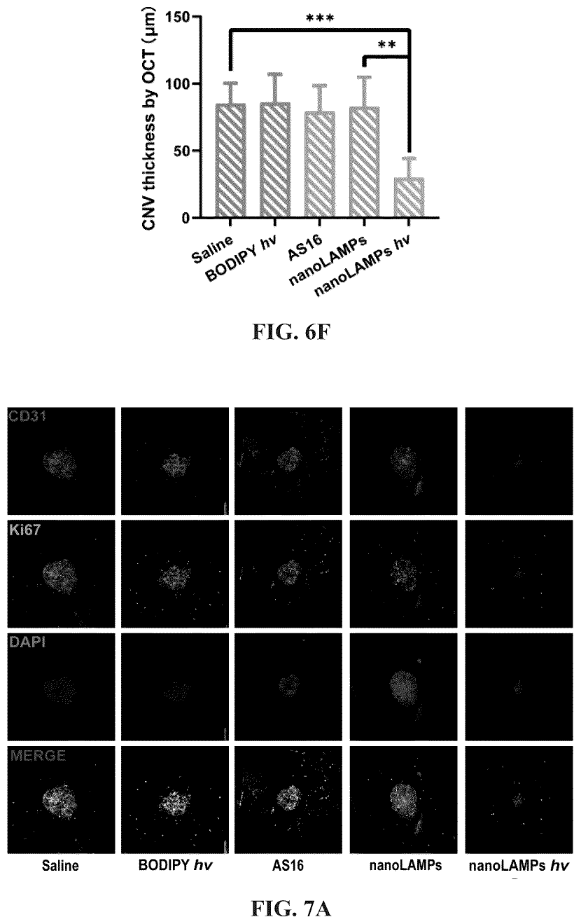

FIGS. 6A-F. Therapeutic efficacy of nanoLAMPs investigated by imaging diagnostics. (A) Representative images of OCTA (optical coherence tomography angiography) of each group. The bright signal located about the optic nerve represents the CNV lesions on the posterior segments of mouse eyes (n=6). (B) FFA (Fluorescence fundus angiography) images of each group taken by Phoenix Micron IV system at 5 min after intravenous injections of fluorescein sodium (n=6). (C) Representative images of OCT (optical coherence tomography) of each group acquired by TowardPi cutting-edge swept source OCT (n=6). (D) Statistical analysis of CNV area by OCTA. (E) Statistical analysis of FFA of each group. Grade I: no hyper fluorescence; Grade II: hyper fluorescence without leakage; Grade III: hyper fluorescence in the early or mid-transit images and late leakage; Grade IV: bright hyper fluorescence that increased in intensity and size. (F) Statistical analysis of CNV thickness of each group. (*: p<0.05; **: p<0.01; ***: p<0.001). The data are presented as mean±SEM.

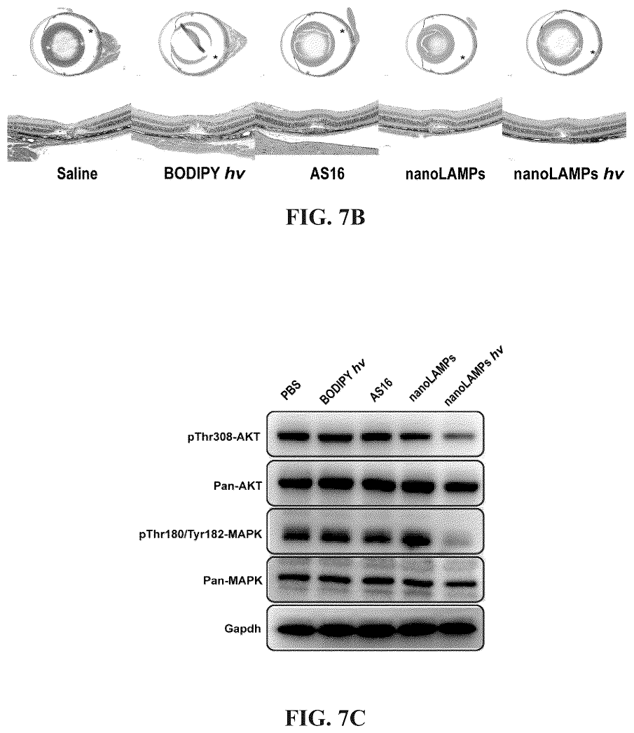

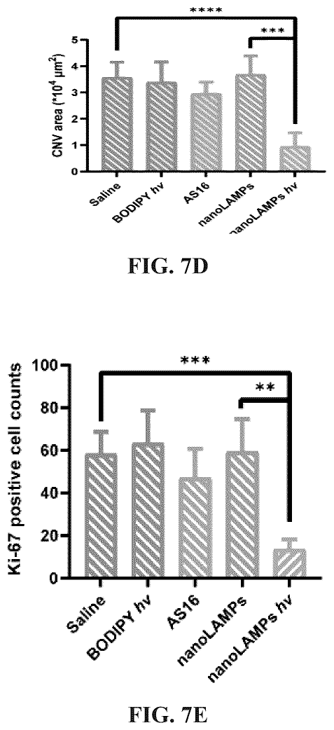

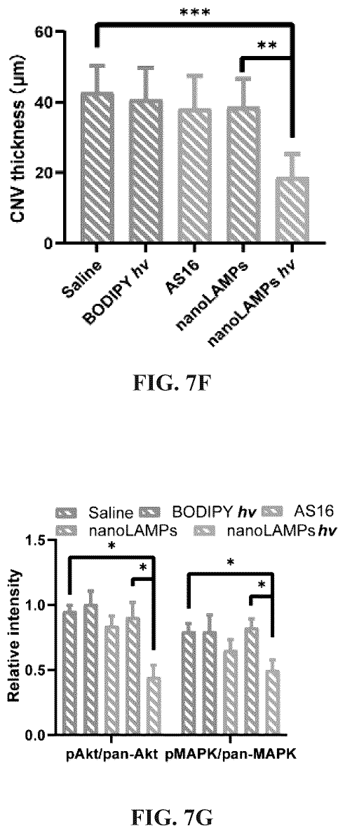

FIGS. 7A-G. Anti-angiogenic efficacy of nanoLAMPs revealed by investigations of isolated tissues. (A) representative images of choroidal flatmounts incubated with CD31 antibody and Ki-67 antibody (n=6). (B) H&E images of each group, the CNV lesions were marked with stars (n=6). (C) Western blot strips for investigations of anti-angiogenesis signaling after each treatment (n=3). (D) Statistical analysis of CNV size marked by CD31 antibody in choroid flatmounts. (E) Statistical analysis of Ki-67 positive cells of each group. (F) Statistical analysis of CNV thickness by H&E sections. (G) Statistical analysis of western blot analysis after each treatment. (*: p<0.05; **: p<0.01; ***: p<0.001. The data are presented as mean±SEM.

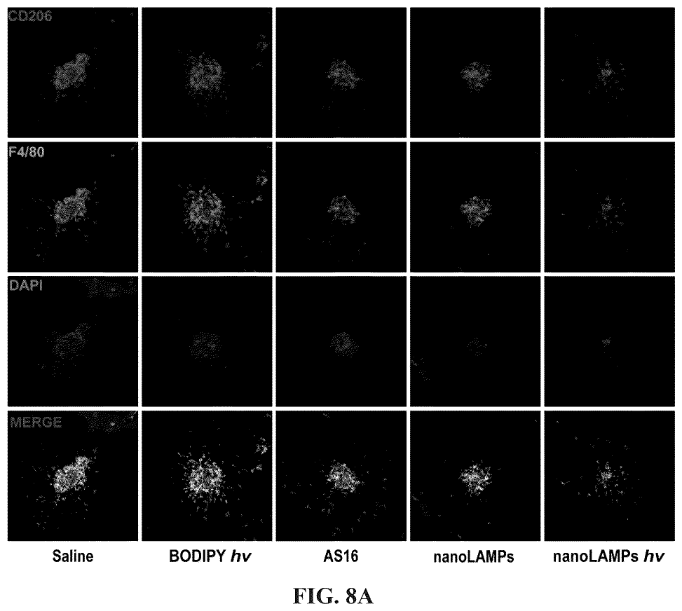

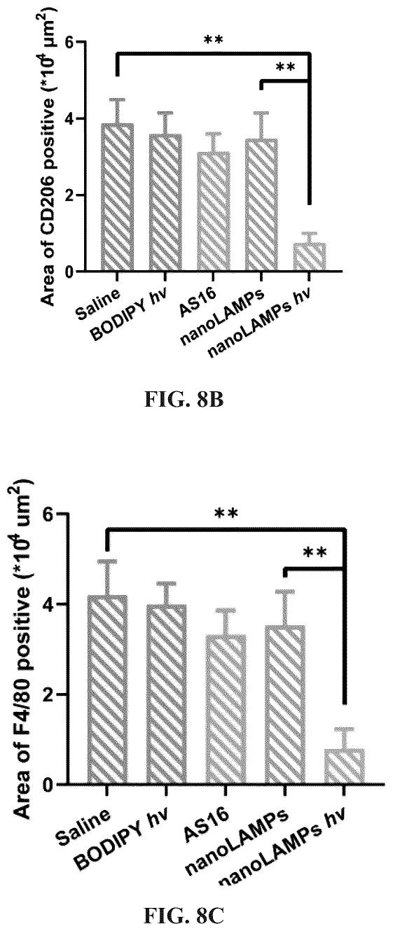

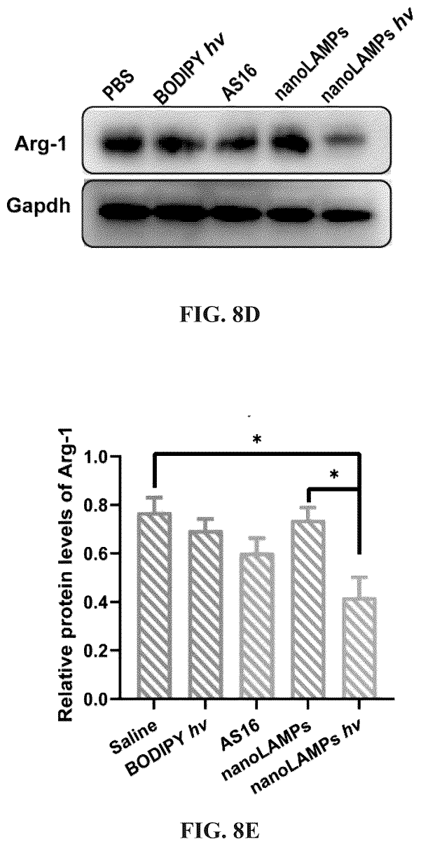

FIGS. 8A-E. Anti-M2 macrophage polarization property of nanoLAMPs. (A) representative images of choroidal flatmounts stained with CD206 antibody and F4/80 antibody (n=6). (B) Statistical analysis of CD206 positive area of each group. (C) Statistical analysis of F4/80 positive area of each group. (D) Western blot analysis trips of Arg-1 and GAPDH (n=3). (E) Statistical analysis of western blot analysis of Arg-1 after each treatment. (*: p<0.05; **: p<0.01). The data are presented as mean±SEM.

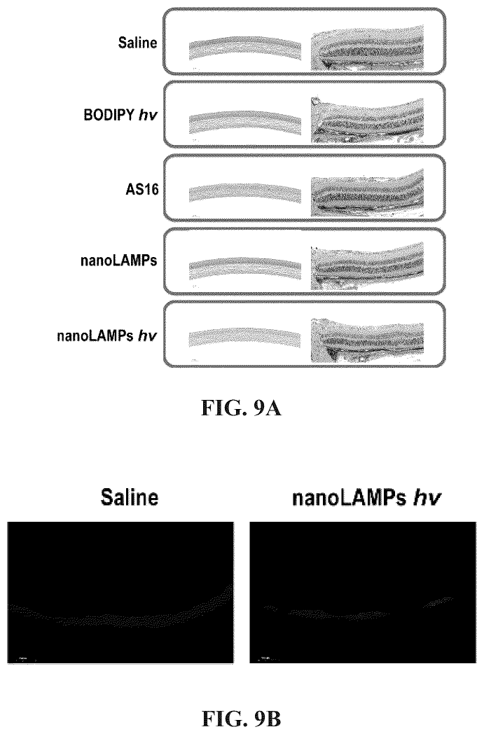

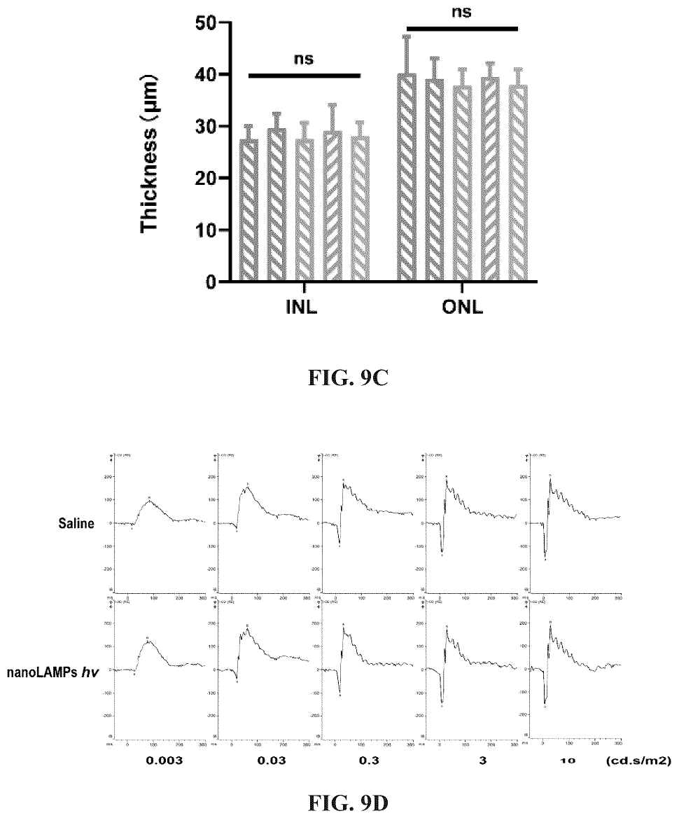

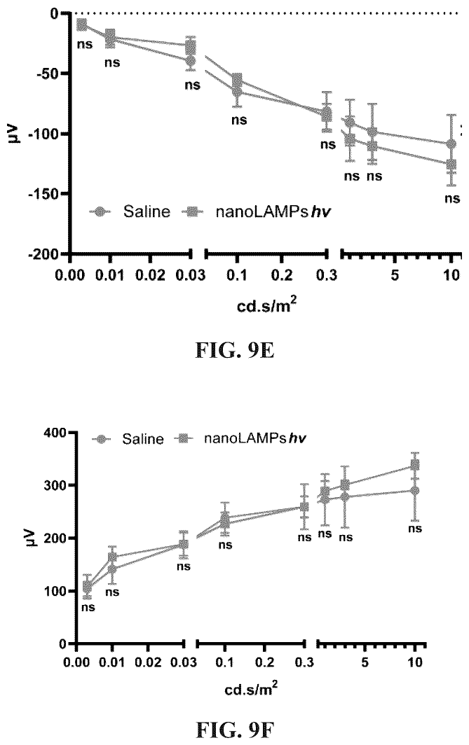

FIGS. 9A-F. Ocular safety profile of nanoLAMPs treatment. (A) Representative images of cornea and retina (close to optic nerve) of each group (n=3). (B) TUNEL images of control group and nanoLAMPs treatment group (n=3). (C) Statistical analysis of INL and ONL thickness of each group. (D) Representative images of fERG (flash electroretinogram) of each group at selected stimuli intensity (n=3). (E) Statistical analysis of scotopic a-wave of each group. (F) Statistical analysis of scotopic b-wave of each group. (ns: not significant). The data are presented as mean±SEM.

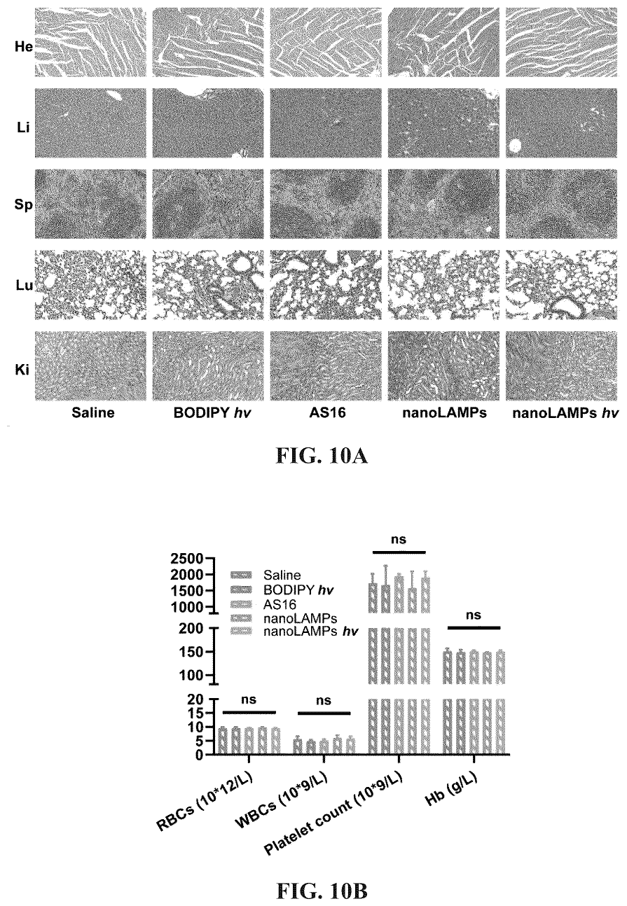

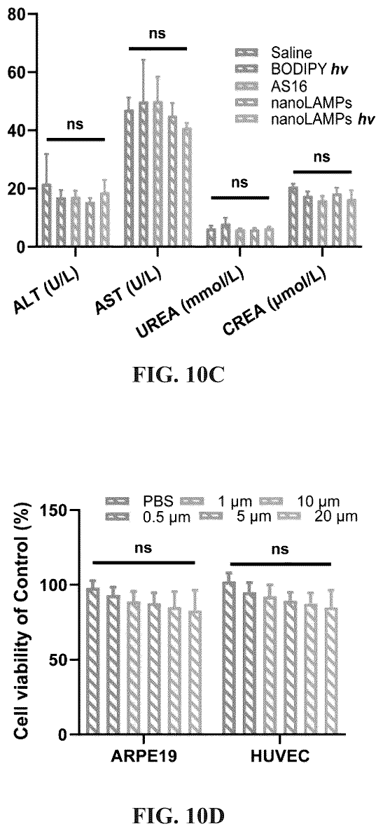

FIGS. 10A-E. Systemic safety profile of nanoLAMPs treatment. (A) representative images of main organs of each group (n=3). (B) Hematological indicators of each group after treatment (n=3). (C) biochemical blood profiles of mice after different formulation treatments (n=3). (D) Statistical analysis of MTT assay of ARPE-19 and HUCECs of each group under dark conditions. (ns: not significant). (E) Body Weight comparing saline, BODIPY, AS16, LAMP treatment over 15 days observation. The data are presented as mean±SEM.

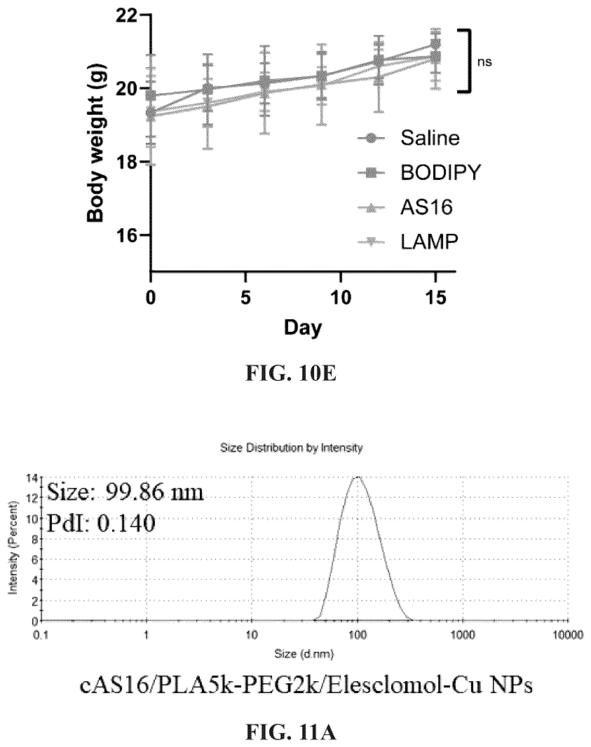

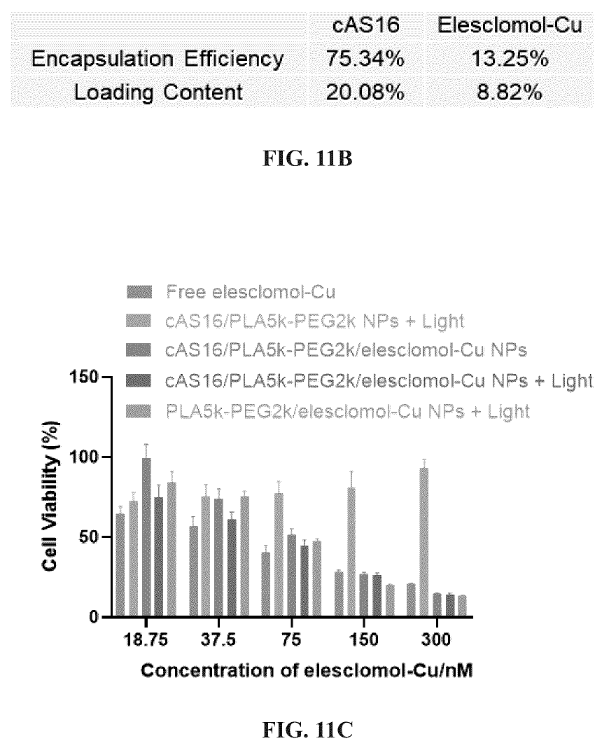

FIGS. 11A-C. Preparation of BODIPY-caged AS16 (cAS16)/PLA5k-PEG2k/elesclomol-Cu NPs. (A) Size distribution of the nanoparticles. (B) Encapsulation efficiency and loading content of cAS16 and elesclomol-Cu. (C) Cytotoxicity of free elesclomol-Cu, cAS16/PLA5k-PEG2k NPs (w/light irradiation), cAS16/PLA5k-PEG2k/elesclomol-Cu NPs (w/or w/o light irradiation), and PLA5k-PEG2k/elesclomol-Cu NPs (w/light irradiation). Light source: LED, 656 nm, 100 mW/cm², n=3, mean±SD.

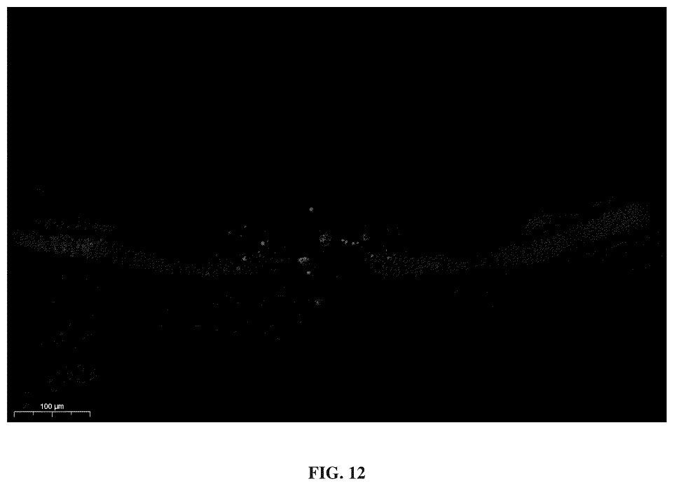

FIG. 12. Tunnel image of CNV lesions. Red fluorescence indicates cell apoptosis.

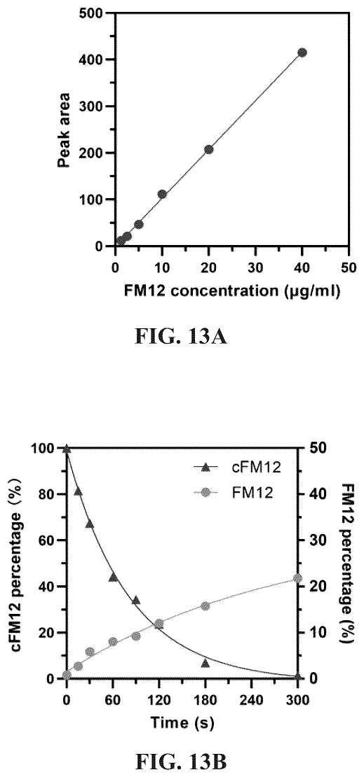

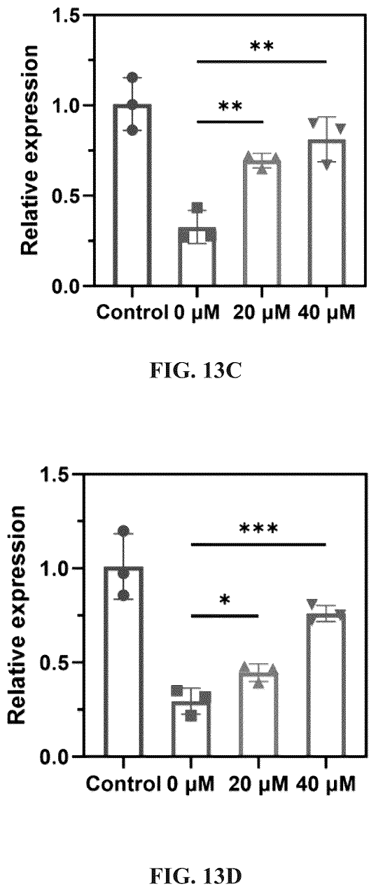

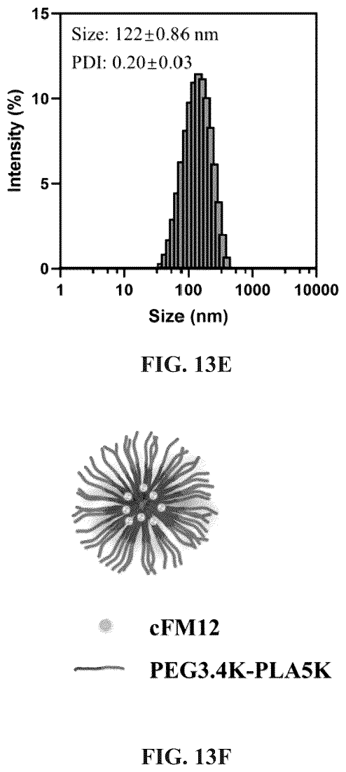

FIGS. 13A-F. BODIPY-caged FM12 peptide (cFM12) nanoparticles for photo-triggered PD-L1 blockade. (A) Standard curve of FM12 peptide as demonstrated by HPLC. (B) FM12 peptides release rate from the nanoparticles triggered by 656 nm light irradiation at consecutive time points as revealed by HPLC. (C) Changes in gene expression of IL-2 by Jurkat T cells treated with different concentrations of cFM12 nanoparticles as revealed by RT-qPCR (n=3). (D) Changes in gene expression of TNF-α by Jurkat T cells treated with different concentrations of cFM12 nanoparticles as revealed by RT-qPCR (n=3). (E) Size distribution of nanoparticles. (F) Schematic diagram of cFM12 nanoparticles. (*: p<0.05; **: p<0.01; ***: p<0.001). The data are presented as mean±SEM.

### 5. DETAILED DESCRIPTION

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% about the value (X±10%). In other contexts the term “about” provides a variation (error range) of 0-10% about a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

As used herein, a “pharmaceutical” refers to a compound manufactured for use as a medicinal and/or therapeutic drug.

As used herein, the term “pharmaceutically acceptable” means compatible with the other ingredients of a pharmaceutical composition and not deleterious to the recipient thereof.

As used herein, a “photoresponsive prodrug” is a compound that can be converted by light irradiation to a specific drug compound or a pharmaceutically acceptable salt of such a compound. Preferably, photoresponsive prodrugs are compounds that are covalently bonded to a photocleavable group.

As used herein, a “photoresponsive group” or “photocleavable group” is a chemical group that can be removed or separated with light irradiation via photocleavage reaction.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Photoresponsive Prodrug

The presently disclosed subject matter includes a photoresponsive prodrug. In particular, in certain embodiments, the photoresponsive prodrug comprises an active agent conjugated to a photoresponsive group. Upon light irradiation, the photoresponsive prodrug will be cleavage, consequently facilitating the release of the active agent. In some embodiments, the photoresponsive prodrugs can respond to NIR light with deeper penetration and less photoxicity to retina in comparison with short-wavelength light.

Active agents of the present disclosure, such as proteins, include but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, viruses, virus vectors, and prions.

In some embodiment, the active agent of the present disclosure is an anti-angiogenic agent. As used herein, an “anti-angiogenic agent” refers to a compound which blocks, or interferes with to some degree, the development of blood vessels. An anti-angiogenic agent may, for instance, be a small molecule, peptide or antibody that binds to a growth factor or growth factor receptor involved in promoting angiogenesis. In some embodiments, an anti-angiogenic agent is a peptide that inhibits VEGF signaling. In some embodiments, an anti-angiogenic agent is a peptide that inhibits M2 macrophage polarization. In some embodiment, an anti-angiogenic agent is a peptide that inhibits both VEGF signaling and M2 macrophage polarization. In some embodiment, an anti-angiogenic agent is AS16 peptide, which consists of 16 amino acids ATWLPPRAANLLMAAS (SEQ ID NO: 1).

In some embodiments, the active agent of the present disclosure targets programmed death ligand 1 (PD-L1). In some embodiments, the active agent is FM12 peptide consisting of amino acid sequence FPNWSLRPMNQM (SEQ ID NO: 2).

In some embodiment, the photoresponsive group comprises a boron-dipyrromethane (BODIPY) unit. As used herein, the term “BODIPY” refers to a structural subunit having the following boron-dipyrromethane (BODIPY) core structure:

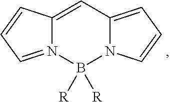

wherein each R is independently selected from the group consisting of F, OH, H, alkyl, substituted alkyl, aryl, substituted aryl, heteroaryl, substituted heteroaryl, alkoxy, substituted alkoxy, alkynyl and substituted alkynyl.

In some embodiments, the photoresponsive group comprises a BODIPY unit of the following formula:

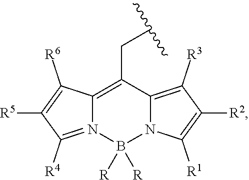

wherein:
the wavy line represents the attachment point of the photoresponsive group; R¹, R², R³, R⁴, R⁵, and R⁶ are each, independently, hydrogen, halogen, optionally substituted C₁-C₆-alkyl, optionally substituted C₂-C₆-alkenyl or (C₆-C₁₄-aryl)-(C₂-C₆-alkenyl-), wherein the (C₆-C₁₄-aryl)-(C₂-C₆-alkenyl-) is optionally substituted on the C₂-C₆-alkenyl or the C₆-C₁₄-aryl. In some embodiments, R¹, R², R³, R⁴, R⁵, and R⁶ are each, independently, hydrogen, halogen, optionally substituted C₁-C₆-alkyl, optionally substituted C₂-C₆-alkenyl, (C₆-C₁₄-aryl)-(C₂-C₆-alkenyl-), wherein the (C₆-C₁₄-aryl)-(C₂-C₆-alkenyl-) is optionally substituted on the C₂-C₆-alkenyl or the C₆-C₁₄-aryl or optionally substituted (C₂-C₈-heterocyclyl-), and
each R is independently selected from the group consisting of F, OH, H, alkyl, substituted alkyl, aryl, substituted aryl, heteroaryl, substituted heteroaryl, alkoxy, substituted alkoxy, alkynyl and substituted alkynyl.

As used herein, the term “C₁₋₆-alkyl” refers to monovalent saturated aliphatic hydrocarbyl groups having from 1 to 6 carbon atoms. This term includes, but is not limited to, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term C₁₋₆-alkyl also includes cycloalkyl groups including, but not limited to, cyclopropyl, cyclobutyl, cyclopentyl, and cyclohexyl.

The term “C₁-C₂₀-alkylenyl” as used herein refers to straight chain and branched, saturated divalent groups having from 1 to 20 carbon atoms, 10 to 20 carbon atoms, 12 to 18 carbon atoms, 1 to about 20 carbon atoms, 1 to 10 carbons, 1 to 8 carbon atoms, 1 to 6 carbon atoms or 2 to 4 carbon atoms. Examples of straight chain C₁-C₂₀-alkylenyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched (C₁-C₂₀)-alkylenyl groups include CH(CH₃)CH₂— and —CH₂CH(CH₃)CH₂—.

As used herein, the term “C₂₋₆-alkenyl” refers to monovalent and divalent unsaturated hydrocarbyl groups having from 2 to 6 carbon atoms. This term includes, but is not limited to, linear and branched hydrocarbyl groups such as vinyl (CH₂═CH—), propenyl (CH₂—CH₂CH₂—), isopropenyl ((CH₃)(CH₂)C—), —CH═CH—, —CH═CH—CH₂—, —CH═CH—CH═CH—, and the like. The term C₂₋₆-alkenyl also includes cycloalkenyl groups including, but not limited to, cyclopentenyl and cyclohexenyl.

As used herein, the term “C₆-C₁₄-aryl” refers to cyclic aromatic hydrocarbons having from 6 to 14 carbon atoms (e.g., 6 to 12 carbon atoms or 6 to 10 carbon atoms). Such aryl groups may be substituted or unsubstituted. Aryl groups include, but are not limited to, phenyl, biphenyl, fluorenyl, phenanthrenyl, and naphthyl groups.

“Substituted” as used throughout the specification refers broadly to replacement of one or more of the hydrogen atoms of a group (e.g., C₁₋₆-alkyl, C₂₋₆-alkenyl, C₆-C₁₄-aryl, and C₂-C₈-heterocyclyl) with substituents known to those skilled in the art and resulting in a stable compound as described herein. Examples of suitable substituents include, but are not limited to, alkyl (e.g., C₁₋₆-alkyl), alkenyl (e.g., C₂₋₆-alkenyl), aryl (e.g., C₆-C₁₄-aryl), alkaryl (e.g., C₁₋₆-alkyl-C₆-C₁₄-aryl), hydroxy, alkoxy (e.g., C₁₋₆-alkyl-O—), aryloxy (e.g., C₆-C₁₄-aryl-O—), carboxy (i.e., CO₂H), alkylcarboxy (e.g., C₁₋₆-alkyl-C(O)O—), arylcarboxy (e.g., C₆-C₁₄-aryl-C(O)O—), cyano, cyanate ester (i.e., an —OCN group), silyl, siloxyl, phosphine, halogen (e.g., F, Cl, Br, and I), nitro, and C₂-C₈-heterocyclyl-. Other suitable substituents include-N(R⁸)₂, wherein each R⁸ is hydrogen, alkyl (e.g., C₁₋₆-alkyl), aryl (e.g., C₆-C₁₄-aryl) or alkaryl (e.g., C₁₋₆-alkyl-C₆-C₁₄-aryl), wherein each alkyl, aryl or alkaryl can be substituted; and —[O—R⁹—]ₚO—R¹⁰, wherein R⁹ is alkylenyl (e.g., C₁-C₂₀-alkylenyl) or cycloalkylenyl (e.g., (C₃-C₂₀)-cycloalkylenyl), R¹⁰ is alkyl (e.g., C₁₋₆-alkyl), and p is an integer from 1 to about 10 (e.g., an integer from 1 to 5, 2 to 8, 2 to 5 or 2 to 4).

In some embodiments, R¹ and R⁴ are each, independently, (C₆-C₁₄-aryl)-(C₂-C₆-alkenyl-), wherein the (C₆-C₁₄-aryl)-(C₂-C₆-alkenyl-) is optionally substituted on the C₂-C₆-alkenyl or the C₆-C₁₄-aryl; that is, the (C₆-C₁₄-aryl)-(C₂-C₆-alkenyl-) is optionally substituted on the C₂-C₆-alkenyl, the C₆-C₁₄-aryl or both. In some embodiments, R¹ and R⁴ are each, independently, (C₆-C₁₀-aryl)-(C₂-C₄-alkenyl-), wherein the (C₆-C₁₀-aryl)-(C₂-C₄-alkenyl-) is optionally substituted on the C₂-C₄-alkenyl or the C₆-C₁₀-aryl; that is, the (C₆-C₁₀-aryl) C₂-C₄-alkenyl-) is optionally substituted on the C₂-C₄-alkenyl, the C₆-C₁₀-aryl or both. In other embodiments, R¹ and R⁴ are each —CH═CH—C₆-C₁₀-aryl, wherein the C₆-C₁₀-aryl is optionally substituted. In still other embodiments, R¹ and R⁴ are each —CH═CH-phenyl, wherein the phenyl is optionally substituted. In still other embodiments, R¹ and R⁴ are each —CH═CH-phenyl, wherein the phenyl is optionally substituted with methoxyl.

In some embodiments, R² and R⁵ are each, independently, hydrogen, halogen, or an optionally substituted C₁-C₆-alkyl. In some embodiments, R² and R⁵ are each, independently, hydrogen, methyl or ethyl. In still other embodiments, R² and R⁵ are both hydrogen.

In some embodiments, R³ and R⁶ are each, independently, an optionally substituted C₁-C₆-alkyl. In some embodiments, R³ and R⁶ are each methyl.

In some embodiments, each R is, independently, F or an optionally substituted C₁-C₆-alkyl. In some embodiments, each R is F. In some embodiments, each R is methyl.

In some embodiments, the photoresponsive group is a moiety selected from formula (VII), formula (VIII), formula (IX), formula (X), formula (XI), or formula (XII):

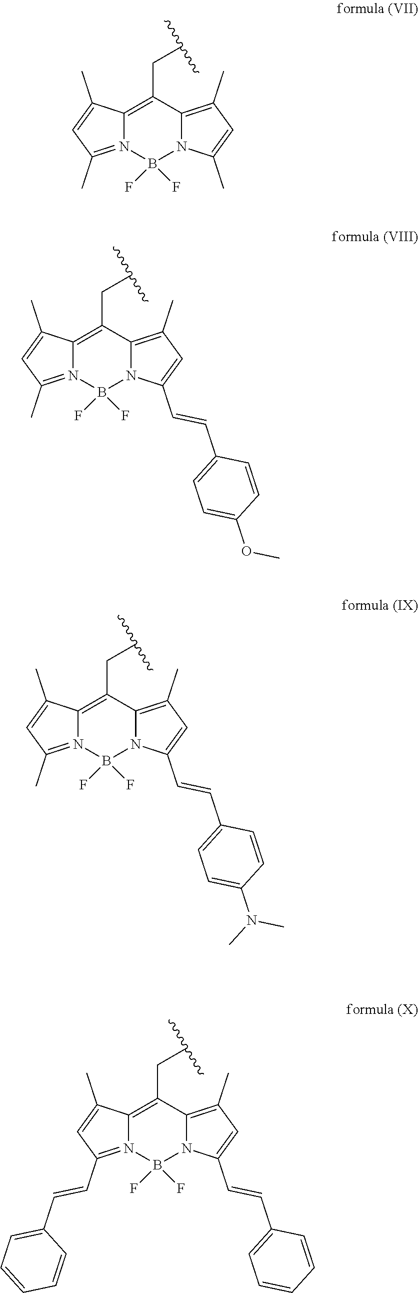

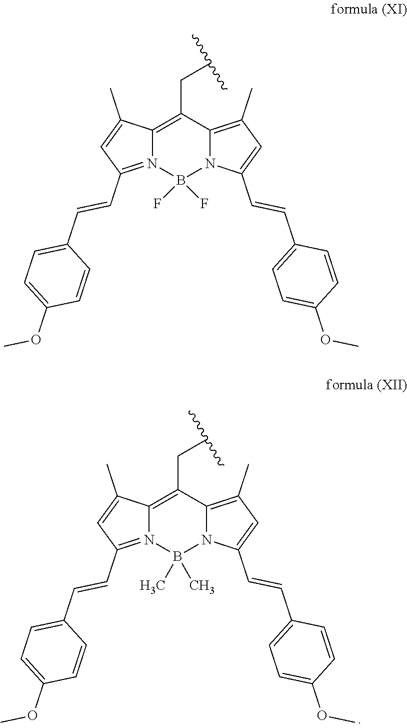

In some embodiments, the photoresponsive group is of formula (XII):

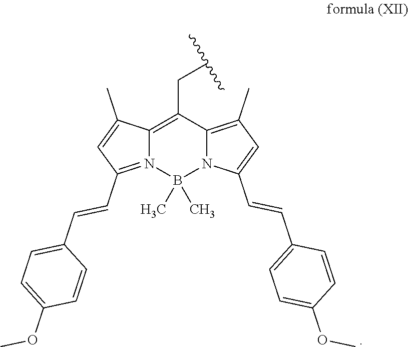

In some embodiments, the active agent is conjugated to the photoresponsive group via a covalent bond, —O—, —NR′—, —S—, —C(═O)—, —C(═O)NR′—, —C(═O)O—, —NR′C(═O)NR′—, —O(C═O)NR′—, optionally substituted C₁-C₆ alkynenyl, optionally substituted C₂₋₆-alkenyl, or a combination thereof, wherein R′ is each independently selected from the group consisting of hydrogen, halogen, OH, H, alkyl, substituted alkyl, aryl, substituted aryl, heteroaryl, substituted heteroaryl, alkoxy, substituted alkoxy, alkynyl and substituted alkynyl. In some embodiments, the active agent is conjugated to the photoresponsive group via a covalent bond, —O—, —NR′—, —C(═O)NR′—, —C(═O)O—, —O(C═O)NR′—, or a combination thereof. In further embodiments, the active agent is conjugated to the photoresponsive group via —O(C═O)NR′—.

In some embodiments, the photoresponsive prodrug of the present invention is of the following formula (LAMP):

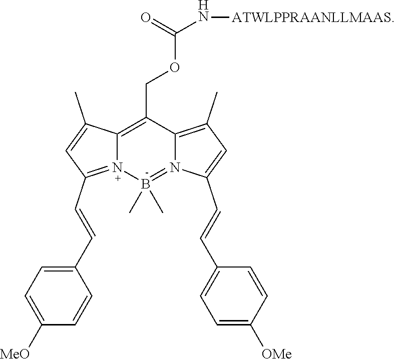

In some embodiments, the photoresponsive prodrug of the present invention is cFM12:

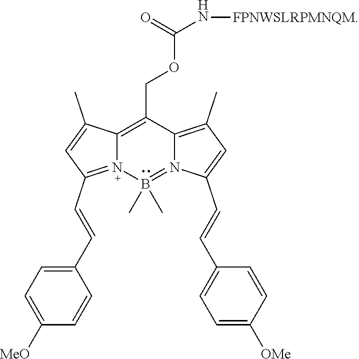

In some embodiments, the photoresponsive prodrugs can be tuned to be light-activated at a particular wavelength and/or over a given range of wavelengths. In some embodiments, the photoresponsive prodrugs can be tuned to be light-activated at certain wavelengths by appropriately selecting the phosphoresponsive group that is included in the compound.

In some embodiments, the photoresponsive prodrug comprises an active agent, and the compound can remain in an inert state until activated by light having a particular wavelength, thereby cleaving the active agent from the photoresponsive prodrug.

As used herein, the term “light” is used herein to refer to any electromagnetic radiation that can activate a compound. In some embodiments light includes ultraviolet light, visible light, near infrared light (NIR), or infrared light (IR). Compared with other kinds of stimuli, light can be utilized to spatiotemporally control drug release, thereby improving therapeutic efficacy and reducing adverse events. The fundus has significantly easier access to the external light irradiations due to the transparent nature of the cornea, lens, and vitreous body. Some embodiments of photoresponsive prodrugs have the surprising and unexpected advantage of being photo-activated by light having wavelengths greater than 500 nm. Other embodiments of the compounds of the present disclosure can be photo-activated by light having wavelengths greater than 650 nm.

More specifically, as used herein, light can refer to energy having a wavelength of about 350 nm to about 1300 nm. In specific embodiments, light can refer to energy having a wavelength of about 500 nm to about 1300 nm. In some embodiments, the light comprises a wavelength of about 500 nm to about 1000 nm. In some embodiments, the light comprises a wavelength of about 600 to about 900 nm. In some embodiments, the light comprises a wavelength of about 600 to about 700 nm. In some embodiments, the light comprises a wavelength of about 656 nm.

### Photoresponsive Nanoparticles

In some embodiments, the photoresponsive prodrugs of the present invention can be self-assembled to drug delivery polymers to form nanoparticles. In some embodiments, the nanoparticles can be prepared by nanoprecipitation. The nanoparticles can protect and guide the drugs, such as AS16 peptides, to the target region to enhance its therapeutic efficacy and silent the therapeutic effect in other tissues and organs, where no light irradiation is applied.

In some embodiments, the polymers to form nanoparticles of the present invention can be selected from: protein-based polymers, such as collagen, albumin, gelatin; polysaccharides, such as agarose, alginate, carrageenan, hyaluronic acid, dextran, chitosan, cyclodextrins; polyesters, such as poly(lactic acid), poly(glycolic acid), poly(hydroxy butyrate), poly(□-caprolactone), poly(β-malic acid), poly(dioxanones); polyanhydrides, such as poly(sebacic acid), poly(adipic acid), poly(terphthalic acid); polyamides, such as poly(imino carbonates), polyamino acids; phosphorous-based polymers, such as polyphosphates, polyphosphonates, polyphosphazenes; other synthetic biodegradable polymers such as poly(cyano acrylates), polyurethanes, polyortho esters, polydihydropyrans, polyacetals; cellulose derivatives, such as carboxymethyl cellulose, ethyl cellulose, cellulose acetate, cellulose acetate propionate, hydroxypropyl methyl cellulose; silicones, such as polydimethylsiloxane, colloidal silica; acrylic polymers, such as polymethacrylates, poly(methylmethacrylate), poly hydro(ethyl-methacrylate); polyvinyl pyrrolidone; poloxamers; poloxamines; and the combinations thereof.

In some embodiments, the polymers to form the nanoparticles of the present invention are selected from polylactic acid (PLA), polyethylene glycol (PEG), polydimethylsiloxane (PDMS), polyethyleneimine (PEI), polyamidoamine (PAMAM), and a combination thereof; preferably, the polymer is selected from PLA-PEG copolymer; more preferably, the polymer is PLA5k-PEG2k.

In some embodiments, the nanoparticles of the present invention are formed by self-assembling the LAMP with PLA5k-PEG2k (nanoLAMPs). The 3D structure and function mechanism of nanoLAMPs are presented as FIG. 1a. After administration, the AS16 peptides will be released from nanoLAMPs on the site of NIR light irradiation and binds to the VEGF receptors and tyrosine kinase receptor 2 (TIE-2) on cell membranes, consequently inhibiting angiogenesis and M2 macrophage polarization.

In some embodiments, the nanoparticles of the present invention are formed by co-assembling cFM12 with PEG3.4k-PLA5k. The schematic diagram of such nanoparticle is shown in FIG. 13f.

### Pharmaceutical Compositions and Kits

The presently disclosed subject matter further includes pharmaceutical compositions comprising the photoresponsive prodrugs or the nanoparticles as disclosed herein. Such pharmaceutical compositions may comprise at least one pharmaceutically acceptable carrier. In this regard, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulation agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

The prodrugs or nanoparticles can also be formulated as a preparation for implantation or injection. Thus, for example, the prodrugs or nanoparticles can be formulated with suitable polymeric or hydrophobic materials (e.g., as an emulsion in an acceptable oil) or ion exchange resins, or as sparingly soluble derivatives (e.g., as a sparingly soluble salt).

The presently disclosed subject matter further includes a kit that can include the photoresponsive prodrugs, the nanoparticles, or the pharmaceutical composition as described herein, packaged together with a device useful for administration. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

### Methods of Use

In certain embodiments, a prodrug and nanoparticles, such as, for example, LAMPS or nanoLAMPS can be administered to a subject. Any means of administration that can permit a nanoparticle to contact cells, including, for example, orally, intravenously, intraperitoneally, intramuscularly, intrathecally, or subcutaneously are envisioned in the subject methods.

As used herein, the term “administration” refers to any method of providing a compound and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In some embodiments, after administration, the photoresponsive prodrugs can release active agent, such as AS16 peptids, upon light irradiation of the subject. In some embodiments, the wavelength of the light can be about 100 nm to about 1000 nm, about 500 nm to about 900 nm, about 620 nm to about 750 nm, or, preferably, about 656 nm. In some embodiments, the subject, nanoparticle, and/or prodrug can be irradiated for about 1 s to about 24 hours, about 10 s to about 12 hours, about 15 s to about 1 hour, about 30 s to about 30 min, about 45 s to about 10 min, or about 7 min. In some embodiments, an entire subject can be irradiated. Alternatively, a specific portion of a subject can be irradiated, such as, for example, eye, head, face, leg, arm, wrist, chest, abdomen, neck, or calf. In some embodiments, the subject can be irradiated before, during, or after administration of the prodrugs, nanoparticles or compositions of the subject invention. In some embodiments, the irradiation of the prodrug, nanoparticle and/or subject can occur at least 1 s, 2 s, 5 s, 10 s, 15 s, 30 s, 45 s, 1 min, 2 min, 5 min, 10 min, 15 min, 30 min, 45 min, 1 hour, 2 hours, 5 hours, or 10 hours after administration of the compounds or compositions of the subject invention. In some embodiments, the subject, nanoparticle, and/or prodrug can be irradiated after the prodrug is at a specific location, such as, for example, tumor, organ, including an eye, or tissue. In certain embodiments, the irradiance can be about 1 mW/cm² to about 1000 mW/cm², 5 mW/cm² to about 500 mW/cm², 10 mW/cm² to about 250 mW/cm², 15 mW/cm² to about 150 mW/cm², 25 mW/cm² to about 125 mW/cm², or about 100 mW/cm². The light can be delivered by any natural or artificial source capable of providing light at the wavelength and irradiance for the described amount of time, including, for example, laser, incandescent, halogen, fluorescent, or light emitting diode (LED).

In some embodiments, the subject prodrugs, nanoparticles or compositions can be irradiated to photoactivate the photoresponsive group, such as, for example, BODIPY. In some embodiments, the irradiation of the BODIPY can generate the cleavage of the photoresponsive prodrug. The cleavage can trigger a cascaded anti-angiogenetic agent release, leading to combined antiangiogenic therapy against CNV.

In certain embodiments, the prodrug and/or nanoparticle is non-toxic before activation by light. Upon light irradiation of the prodrug and/or nanoparticle, preferably at 656 nm, an anti-angiogenetic agent can be released from the prodrug or composition containing the nanoparticles or the prodrug.

Still further, the presently disclosed subject matter includes a method for treating diseases, such as cancer and age-related macular degeneration (AMD). In some embodiments, the method comprises administering a prodrug, a nanoparticle, or a composition as disclosed herein, and then exposing the administration site of the subject to light after the compound has been administered. As described above, the light in some embodiments can be a light having a wavelength of about 500 nm to about 1300 nm.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Materials and Methods

Reagents. The AS16 peptides were synthesized and purchased from Genescript (Nanjing, China). The PLA5k-PEG2k was purchased from Ponsure Biotech (Shanghai, China). The human umbilical vein endothelial cells (HUVEC) and human retinal pigment epithelial cells (ARPE-19) were obtained from American Type Culture Collection (Manassas, VA, USA). Gibco Dulbecco's Modified Eagle medium (DMEM), fetal bovine serum (FBS), penicillin-streptomycin solution and trypsin-EDTA 0.25% solution, collagen I (rat tail) were purchased from Thermo Fisher Scientific, Inc. (Eugene, OR, USA). BCA Protein Assay Kit were purchased from Thermo Fisher Scientific, Inc. (Eugene, OR, USA). Primary antibodies involved in this investigation are summarized in Table1.

TABLE.1
Summary of primary antibodies used in this investigation.

| Antibody | Company | Cat no. | Dilution |
|---|---|---|---|
| CD31 | Millipore | MAB1398Z | 1:200 |
| | (Massachusetts, | | |
| | USA) | | |
| Ki-67 | CST (California, | 9192S | 1:100 |
| | USA) | | |
| F4/80 | Servicebio | GB113373 | 1:100 |
| | (Wuhan, China) | | |
| CD206 | BioRAD | MCA2235GA | 1:100 |
| | (California, USA) | | |
| AKT | Abcam (Cambridge, | ab8805 | 1:1000 |
| | UK) | | |
| pThr308-Akt | Abcam (Cambridge, | ab38449 | 1:1000 |
| | UK) | | |
| MAPK | ABclonal (Wuhan, | A14401 | 1:1000 |
| | China) | | |
| pThr180-Tyr182-MAPK | ABclonal (Wuhan, | AP0526 | 1:1000 |
| | China) | | |
| Arg-1 | Servicebio | GB11285 | 1:1000 |
| | (Wuhan, China) | | |
| GAPDH | CST (California, | 2118S | 1:1000 |
| | USA) | | |

Instruments. Dynamic light scattering (DLS) analysis was conducted by Nano-ZS (Malvern Instruments, UK). TEM images were captured by CM100 Transmission Electron Microscope (Philips, USA). UV-vis absorption spectra and cell viability were measured by SpectraMax M4 multi-mode microplate reader (Molecular Devices, USA). Drug-loading capacity, encapsulation efficiency, and drug concentrations were detected by 1260 Infinity II HPLC (Agilent Technologies, USA). Photolysis experiments and irradiation were conducted using a commercial diode laser system (Laserwave, Canada). The light irradiance was measured by PM100USB power and energy meter (Thorlabs, USA) equipped with S142C integrating sphere photodiode power sensor (Si, 350-1100 nm, Thorlabs, USA). Fluorescence images were captured by LSM 980 confocal microscope (ZEISS, German). Experimental CNV mouse model was constructed using OcuLight Infrared Laser System (IRIDEX, USA). OCTA, OCT images were captured by TowardPi cutting-edge swept source OCT (TowardPi Medical Technology Ltd, China). FFA images were captured by Phoenix Micron IV retina imaging system (Phoenix Research Laboratories, USA). ERG results were acquired by Celeris-Diagnosys system (Diagnosys, USA).

Animals. The animal studies were carried out under the approval and supervision of the Committee on the Use of Live Animals in Teaching and Research, Zhongshan Ophthalmic Center, Sun Yat-sen University. C57BL/6J mice (18-22 g, 6˜8 w, male) were obtained from the Shanghai Model Organisms Center (Shanghai, China). All the mice were housed in conventional experimental holding areas with an alternating 12 h light/dark-period control, regulated temperature at 21±4° C., relative humidity at 50±10% and ad libitum feeding to UV-treated food and 1-μm-filtered water.

Statistical analysis. All the data were presented as mean±SD except for specific notification. Statistical analysis was performed using GraphPad Prism 9.0.1 software. Unpaired two-tailed student's t-test was used for between-group comparisons, while one-way ANOVA test combined with the Dunnett's post hoc test was utilized for multi-group analysis. p<0.05 was considered as a statistical significance.

### Example 1. Nanoparticle Preparation and Characterization

### 1.1 Synthesis of BODIPY-NPC

The BODIFY-NPC was synthesized according to synthesis route shown in FIG. 1b. 1H spectrum of the resultant BODIPY-NPC is shown in FIG. 2a.

1.1.1 Synthesis of (5,5-Difluoro-1,3,7,9-tetramethyl-5H-4λ4,5λ4-dipyrrolo[1,2-c: 2′,1′-f][1,3,2]diazabo-rinin-10-yl)methyl Acetate (BODIPY-F2-OAc) (A)

2-Chloro-2-oxoethyl acetate (0.6 ml, 5.6 mmol, 1.2 equiv) was added to a solution of 2,4-dimethylpyrrole (1.0 ml, 9.3 mmol, 2.0 equiv) in anhydrous DCM (40 ml) under a nitrogen atmosphere. The reaction was stirred under reflux for 3 h. After this time, DIPEA (3.1 ml, 18.6 mmol, 4 equiv) was added. The resulting mixture was allowed to stir at room temperature for another 30 min. Then boron trifluoride diethyl etherate (2.3 ml, 18.6 mmol, 4 equiv) was added and the reaction solution was stirred for 30 min. Then silica was added to the flask, and the solvents were evaporated. BODIPY-F2-OAc was purified by flash chromatography with the CombiFlash® system. The product was obtained as red-gold crystals (814 mg, 45.4% yield).

1.1.2 Synthesis of (5,5-difluoro-3,7-bis ((E)-4-methoxystyryl)-1,9-dimethyl-5H-4λ4,5λ4-dipyrrolo[1,2-c: 2′,1′-f][1,3,2]diazaborinin-10-yl)methyl acetate. (B)

Compound A (200 mg, 0.652 mmol, 1 equiv) was reacted with 4-anisaldehyde (4 ml, 32.8 mmol, 53 equiv) at 60° C. for 2 h under a nitrogen atmosphere in the dark. The reaction was monitored by TLC. During the period, the color of the mixture changed from red to purple and then to dark green. The mixture was cooled to room temperature and purified by flash chromatography with the CombiFlash® system. The product was obtained as a deep red powder (258 mg, 74.2% yield).

1.1.3 Synthesis of (5,5-difluoro-3,7-bis ((E)-4-methoxystyryl)-1,9-dimethyl-5H-4λ4,5λ4-dipyrrolo[1,2-c:2′,1′-f][1,3,2]diazaborinin-10-yl)methanol. (C)

A mixture of aqueous NaOH solution (43 mg, 0.5 ml, 1.076 mmol, 4 equiv) and methanol (9.5 ml) was dropwise added to a solution of compound B (150 mg, 0.269 mmol, 1 equiv) in DCM. The reaction was monitored by TLC and stirred for 2 h in the dark at room temperature. After this time, the solvent was evaporated and re-dissolved with DCM. Then, the solution was extracted by 0.01 M HCl. The organic layer was collected and hydrated by anhydrous sodium sulfate. The product was obtained as a dark red powder by evaporation without further purification (126 mg, 90.87% yield).

1.1.4 Synthesis of (3,7-bis((E)-4-methoxystyryl)-1,5,5,9-tetramethyl-5H-4λ4,5λ4-dipyrrolo[1,2-c:2′,1′-f][1,3,2]diazaborinin-10-yl)methanol. (D)

Compound C (100 mg, 0.194 mmol, 1 equiv) was dispersed in 5 ml Et2O. Methylmagenesium bromide (3M, tetrahydrofuran (THF) solution, 0.3 ml, 0.97 mmol) was added dropwise to the compound C solution under a nitrogen atmosphere in the dark. The reaction was monitored by TLC. The color of the solution changed from dark green to dark blue. After 2 h stirring, the reaction was stopped by dropwise adding 0.5 ml distilled water to the mixture. THF and Et2O were removed by evaporation. The residue was re-dissolved in DCM. The DCM solution was extracted by saturated sodium chloride aqueous solution three times. The organic layer was collected and dehydrated by anhydrous sodium sulfate. The resulting product was then obtained as a dark blue solid by flash chromatography (48 mg, 48.9% yield).

1.1.5 Synthesis of (3,7-bis((E)-4-methoxystyryl)-1,5,5,9-tetramethyl-5H-4λ4,5λ4-dipyrrolo[1,2-c:2′,1′-f][1,3,2]diazaborinin-10-yl)methyl (4-nitrophenyl)carbonate. (E)

Compound D (40 mg, 0.079 mmol, 1 equiv) was dissolved in anhydrous DCM (2 ml) with DIPEA 80 μl, 0.39 mmol, 5 equiv) and pyridine (30 μl, 0.312 mmol, 4 equiv) at nitrogen atmosphere. 4-nitrophenyl chloroformate (158 mg, 0.78 mmol, 10 equiv) in 1 ml DCM was dropwise added to the compound 3 solution in an ice bath. The reaction was stirred in the dark for 4 h at room temperature. After this time, the mixture was purified by flash chromatography. The product was obtained as a dark powder (41 mg. 77.3% yield).

1.2 Synthesis of BODIPY-AS16 conjugate (LAMP)

20 mg AS16 peptide (1 e.q., 0.012 mmol) was dissolved in 500 μL anhydrous DMSO. 2 μL DIPEA was then added to the AS16 solution. Subsequently, BODIPY-4-NPC (3 e.q., 0.036 mmol) was dissolved in 500 μL anhydrous DCM and dropwise added to the AS16 solution. The mixture was stirred under N₂ protection at room temperature overnight. The DCM was removed by evaporation. The mixture was diluted with ACN and the NIR light-responsive BODIPY-AS16 conjugate was purified by HPLC. ¹H spectrum of the resultant LAMP is shown in FIG. 2b, and the UV-vis spectrum of the resultant LAMP is shown in FIG. 3a. The UV-vis spectrum of LAMP showed characteristic absorption peaks of the AS16 and the BODIPY at 220 nm and 656 nm, respectively, indicating the successful synthesis of the LAMP.

1.3 Preparation of NIR light-responsive BODIPY-AS16 conjugate nanoparticles (nanoLAMPs)

The nanoLAMPs were prepared by nanoprecipitation. Specifically, 4 μL 100 mg/ml PLA5k-PEG2k was mixed with 4 μL 25 mg/ml BODIPY-AS16 conjugate and added to ddH2O during vigorous stirring. The size distribution and zeta-potential were determined by Malvern Zetasizer (Nano ZS90, Malvern, UK). The morphology investigation was conducted with transmission electron microscopy (TEM, Hitachi S-4800, Tokyo, Japan).

The dynamic light scattering (DLS) showed that the size of nanoLAMPs was about 60.74 nm with a polydispersity index (PdI) at about 0.17 as shown in FIG. 3b. The zeta potential of nanoLAMPs was about −1.43 mV.

### Example 2. Colloidal Stability Evaluation of nanoLAMPs

The stability of the nanoLAMPS was evaluated by DLS analysis and TEM imaging after incubation with PBS buffer (pH 7.4) and serum containing DMEM medium (10%) at 37° C. for at least 48 h. The size and morphological changes of nanoparticles can indicate their colloidal stability. The nanoLAMPs showed satisfying stability property in DMEM complete medium containing 10% FBS as detected by DLS since no obvious size and PdI changes were observed in 48 h at 37° C. (FIG. 3c). The encapsulation efficiency and loading capacity of LAMPs in the nanoparticles were found to be 72.69%±2.49% and 21.78%±1.78%, respectively (FIG. 3g).

### Example 3. NIR Light Triggered Drug Release

After 656 nm laser irradiation at 100 mW/cm² for various intervals (0, 1, 2, 5, and 7 min), the photocleavage products released from the nanoLAMP solutions were separated by centrifugation, fully dissolved in acetonitrile/methanol mixture (1:1) and further analyzed by HPLC. The cumulative drug release in the dark was evaluated as the control for drug leakage calibration before illumination. NanoLAMPs degradation rate (%) was calculated as the percentage of decomposed LAMP versa initial LAMP content (t=0). AS16 recovery percentage (%) was identified as the molar ratio of generated AS16 peptide versa the initial AS16 content.

The size change after 656 nm light irradiation was investigated by DLS and transmission electron microscope (TEM). As shown in FIG. 3b, a significant change of nanoparticle size from 60.74 nm to 44.94 nm upon 656 nm light irradiation (100 mw/cm², 5 min) was observed and the TEM results showed consistent results (FIGS. 3b and 3e). The recovery rate of the free AS16 peptides was also detected by HPLC analysis at 220 nm, which showed that about 30% free AS16 peptide was released after light irradiation (FIGS. 3d and 3h). An obvious color change can be observed with naked eye after light irradiation (FIG. 3f). Furthermore, the release of free BODIPY molecule was detected simultaneously at 656 nm (FIG. 4). These results confirmed that nanoLAMPs could respond quickly to NIR light irradiation within 5 min and trigger the release the encapsulated AS16 peptides at the desired spot.

### Example 4. In Vitro Investigation of nanoLAMPs for Anti-Angiogenic Therapy

4.1 Tube formation assay

60 μL of the melted matrix gel was coated on the bottom of the wells of 96-well plates and waited until solidification. Afterwards, 100 μL complete culture medium containing 2*10⁴ HUVEC cells were added into the wells. The wells were divided into 5 groups (PBS, BODIPY plus light irradiation, AS16 peptide, nanoLAMPs, nanoLAMPs plus light irradiation) and received treatment accordingly. The concentration of AS16 peptide was set to 5 μM and the parameter of light irradiation was 656 nm, 100 mw/cm², 5 min. The junction numbers and total segment length of each group were measured by Image J software (1.53e). The data analysis was made by GraphPad Prism software (9.0.0).

### 4.2 Wound Healing Assay

5*10⁵ HUVEC cells were seeded in the 24-well plates and incubated until they reached 90% confluence. Afterwards, a scratch crossing each well was conducted by 200 μL pipette head. The cells were then be washed with PBS for 2 times to remove the flouting cells and incubated with 1 ml DMEM medium with 5% FBS. The grouping and treatment method was consistent with above. The data of each group was measured and analyzed by Image J and GraphPad Prism, respectively.

### 4.3 Migration Assay

200 μL serum-free medium containing 2*10⁴ HUVEC cells were seeded in the upper chamber of the trans-well and 600 μL full medium was loaded in the lower chamber. Different drugs were added to the upper chamber. The grouping and treatment method as well as the calculation was consistent with above.

### 4.4 Results

To verify the anti-angiogenic property of nanoLAMPs in vitro, tube formation assay, migration assay, and wound healing assay were also conducted on HUVECs and the concentration of each group was set equal to nanoLAMPs containing 5 μM AS16 peptide. After NIR light irradiation, the nanoLAMPs exerted similar or even better therapeutic effect to the AS16 only group, with an almost totally inhibited formation of tube-like networks between HUVECs. In the contrary, the nanoLAMPs showed negligible therapeutic effect to the tube formation process in the dark condition, since the AS16 peptide was caged by BODIPY inside the nanoparticles and away from the receptors on the cell surface because of long PEG chains when the NIR light irradiation was absent (FIG. 5a). This result indicated that nanoLAMPs are photo-responsive and able to release functional AS16 peptides upon NIR light irradiation.

Likewise, the migration assay showed similar trend to the tube formation assay, with the least migrated HUVECs in the nanoLAMPs plus light irradiation group and the AS16 peptide group as shown in FIG. 5b. However, when the light was absent, the number of the cells on the lower chamber remained the same as the control group. This result demonstrated that nanoLAMPs successfully inhibited HUVEC migration upon light irradiation while remaining negligible therapeutic effect when the light is absent.

Finally, the wound healing assay showed consistent result, which showed a significant compromised recovery of generated scratches in nanoLAMPs plus light irradiation group and AS16 peptide group (FIG. 5c). The statistical analysis of tube formation, migration and wound healing was conducted by GraphPad Prism software and were shown as FIG. 5f-5i, which presented significant superior anti-angiogenesis property of nanoLAMPs upon light irradiation.

Since the AS16 peptide was previously designed to inhibit VEGF signaling, its therapeutic efficacy was further evaluated by western-blot analysis. According to previous research, the phosphorylation of AKT and MAPK plays as a key role in the cell proliferation and vessel sprouting. As shown in FIG. 5d, the upregulated phosphorylation of AKT and MAPK by VEGF was successfully inhibited by nanoLAMPs with light irradiation, with a significantly decreased phosphorylation of AKT and MAPK proteins, whose trends were similar to the AS16 peptide group. This result indicates that nanoLAMPs successfully released functional AS16 peptide upon

NIR light irradiation and inhibited VEGF signaling and angiogenesis.

### Example 5. Therapeutic Efficacy of nanoLAMPs for Anti-Angiogenic Therapy in Laser Induced CNV Mouse Model by Imageological Examination

5.1 Construction of laser induced CNV mouse model.

Four laser spots were produced on the fundus of mice by 810 nm laser to induce CNV. Specifically, C57BL/6 mice (6-8 w) were anesthetized with 1% pentobarbital sodium (40 mg/kg). Then their pupils were dilated with 0.5% tropicamide eyedrops and received local anesthesia by tetracaine eye drops, followed by carboxymethylcellulose sodium eye drops for prevention of ocular surface dehydration. Each mouse's eyes were applied four laser (six in case of western blot analysis) burns (810 nm wavelength, 140 mW of power, 75 μm of spot size, 75 ms of duration) with OcuLight Infrared Laser System at almost equal distance to the optic nerve head. Afterwards, the mice received chloramphenicol eye drops for infection prevention and rested on heating pad until consciousness. All animal procedures were carried out following the committee's guidelines on the Use of Live Animals in Teaching and Research, Zhongshan Ophthalmic Center, Sun Yat-sen University.

5.2 Treatment of the mice.

The mice with equal size of laser lesions were selected and randomly divided into 5 groups: the saline group, BODIPY plus light irradiation group, AS16 peptide group, nanoLAMPs group and nanoLAMPs plus light irradiation group. On day 3 and day 5 after laser photocoagulation, 2 mg/kg AS16 peptide and quantity equivalent nanoLAMPs were administered intravenously through tail veins. The mice in nanoLAMPs plus light irradiation group received light irradiation into the eyes 1 h after injection (656 nm, 50mw/cm², 5 min) and the group without light irradiation were carefully housed in dark room to avoid the unexpected light source. On day 7 post CNV induction, FFA (fluorescence fundus angiography) and OCTA (optical coherence tomography angiography) images of each group was collected, and the mice were then euthanized. The eyes were enucleated for further investigations.

### 5.3 OCTA.

The Optical coherence tomography angiography (OCTA) images were captured by using Beiming ultra-wide field swept source OCT (TowardPi Medical Technology Ltd). The CNV lesion was defined as a spindle-shaped hyperreflective area, which the direction of long axis was consistent with the level of the RPE. Only the scan that passed through the center of the lesion was used to measure the thickness and length of the CNV lesion, which was analyzed and calculated by using ImageJ software and GraphPad Prism.

### 5.4 FFA

For FFA, the fluorescein sodium was injected intraperitoneally (0.3 mL of 2% fluorescein sodium), and sequential real-time FFA images were captured at 4-5 minutes after fluorescein injection by Pheonix Micron IV. The leakage grades of CNV were evaluated by two specialists independently, and the grading criteria of FFA images were as follows: grade I meant no hyperfluorescence; grade II meant hyperfluorescence without leakage; grade III meant hyperfluorescence in the early or mid-transit images and late leakage; and grade IV meant bright hyperfluorescence that increased in intensity and size during the transit phase of the angiogram.

### 5.5 Results

As shown in FIGS. 6a and 6d, nanoLAMPs with light irradiation significantly decreased CNV area as revealed by OCTA compared with other groups. The AS16 peptide, BODIPY plus light irradiation, as well as nanoLAMPs without light exposure showed negligible effect. Also, the FFA test indicated that the nanoLAMPs plus light irradiation successfully inhibited leakage from CNV lesions, which is a main cause for severe vision loss in exudative AMD patients (FIGS. 6b and 6e). Furthermore, the OCT images showed a similar trend in the thickness of the CNV, with a significantly decreased thickness of more than 70% compared with control group in nanoLAMPs plus light irradiation group (FIGS. 6c and 6f). These results of imageological examination indicated that nanoLAMPs effectively decreased the severity of laser-induced CNV in mice when light irradiation was applied, while AS16 peptides were blocked in the nanoparticles when no light was unavailable.

### Example 6. Therapeutic Efficacy of nanoLAMPs for Anti-Angiogenic Therapy in Laser Induced CNV Mouse Model by Isolated Tissue

Further investigations were conducted to finally determine the treatment efficacy of nanoLAMPs.

6.1 Immunofluorescent staining of choroidal flat mounts.

To confirm the efficacy of neovascular inhibition by nanoLAMPs, CNV mice with relevant treatment were anesthetized, and their eyes were subsequently extracted on day 7 post laser induction. The cornea, lens, and vitreous body are carefully removed after fixing in the 4% paraformaldehyde for 30 min in room temperature and the RPE-choroid-sclera complex was carefully isolated. Afterwards, the ocular tissue was stained with primary antibody at 4° C. overnight. After being washed with PBS buffer six times (5 min for each round) the RPE-choroid-sclera complex were incubated with secondary antibodies for 2 h in room temperature followed by 8 rounds of PBS washing (5 min for each round). Flat-mounted RPE-choroid slides were prepared by making 4 cuts from margin to the optic nerve, and the fluorescence images were acquired by confocal microscopy (LMS980 Carl Zeiss). The CNV lesion area was quantitatively analyzed by ImageJ software.

6.2 Western blot analysis.

After the treatment, choroid tissue from each group was isolated and lysed with RIPA buffer containing 1% of protease and phosphatase inhibitor. The protein samples were normalized by BCA protein assay, loaded in loading buffer and boiled for 10 min (95° C.), and then separated by 5%/12% sodium dodecyl sulfate-polyacrylamide gel electrophoresis (SDS-PAGE). Ander transfer to polyvinylidene difluoride filter (PVDF) membrane under constant current at 250 mA for 1.5 h and being incubated by 5% bovine serum albumin (BSA) blocking buffer for 1.5 h, the protein membranes were incubated with primary antibodies in WB primary antibody diluent (Servicbio, Wuhan, China) in 4° C. overnight followed by 6 rounds of 5 min washing with tris buffered saline Tween (TBST). Horseradish peroxidase (HRP)-secondary antibodies were diluted in TBST and incubated with the strips in room temperature for 2 h, followed by PBS washing. After being reacted with the ECL Western blotting substrate, the protein membranes were visualized by a chemiluminescence imaging system. GAPDH staining was utilized for standardization of the samples.

6.3 Histological assessment.

Enucleated eyeballs and the major organs were collected, fixed with FAS eyeball fixative solution (for eyes)/4% paraformaldehyde (for major organs), and then embedded in paraffin. Visual axis-oriented eyeball sections and histological structural oriented organ sections were prepared and then stained with hematoxylin and eosin (H&E) for histologic examination.

### 6.4 Results.

As shown in FIGS. 7a and 7d, the size of the CNV lesions marked by CD31 antibody decreased dramatically in nanoLAMPs plus light irradiation group compared with other groups, consistent with the other results. Additionally, a significantly decreased Ki-67 positive cells in the CNV lesions with nanoLAMPs administration and light irradiation group was observed compared with other groups, indicating an anti-angiogenesis activity in CNV lesions by nanoLAMPs (FIGS. 7a and 7e). In consistence, a remarkable reduction in the vertical dimensions of CNV was observed in the haematoxylin and eosin (H&E) stained eyeball cross-sections of nanoLAMPs plus irradiation group (FIGS. 7b and 7f). It was worth mentioning that the AS16 peptide group showed negligible therapeutic effect compared with the control group in all the tests in vivo, which was estimated to resulted from the fast clearance and degradation of AS16 peptides in blood flow after systemic administration. More importantly, the nanoLAMPs nanoparticle without light irradiation group also showed no obvious treatment efficacy, indicating that the AS16 peptide was successfully blocked and prevented to effect when light irradiation was kept absent. This phenomenon further confirmed a decreased risk of potential systemic adverse events and undesired therapeutic effect of AS16 peptide by our nanosystem. Moreover, the western-blot analysis showed that the activity of VEGF signaling was successfully inhibited with nanoLAMPs in vivo when the light irradiation is applied (FIGS. 7c and 7g), which showed significantly inhibited phosphorylation of AKT and MAPK proteins. Taken together, these results demonstrated that the nanoLAMPs can effectively inhibit angiogenesis in vivo when the NIR light irradiation is applied, and the photo-responsiveness of nanoLAMPs in vivo was also confirmed.

### Example 7. Anti-M2 Macrophage Polarization Property of nanoLAMPs In Vivo

NanoLAMPs' inhibitory activity on M2 macrophage polarization in vivo was also investigated, since AS16 was reported to successfully inhibit the M2 macrophage polarization by binding to TIE2 receptors, which are previously reported to be involved in macrophage induced angiogenesis. The M2 macrophage was marked with CD206 antibody, and its fluorescent signal of choroidal flat mounts was collected by confocal microscope, with the same protocol of Example 6.1. As shown in FIGS. 8a and 8b, the CD206 positive area in nanoLAMPs nanoparticle with light irradiation group was significantly decreased compared with other groups, indicating the successful inhibition of M2 polarization of macrophages in CNV lesions with our treatment. The CD206 signal exerted good colocalization of F4/80, a well-defined marker for mature and activated macrophages, indicating that M2 macrophage polarization dominates the angiogenic process at late phase of laser induced CNV, consistent with previous literatures. Also, the western blot analysis (with the same protocol of Example 6.2) showed that the expression of Arg-1 protein, defined as the specific marker of M2 macrophage, was significantly inhibited by nanoLAMPs plus light irradiation group compared with other groups (FIGS. 8d and 8e). These results demonstrated that our nanoparticle can effectively and accurately inhibit M2 macrophage polarization in vivo with light irradiation.

### Example 8. In Vitro and In Vivo Biocompatibility of nanoLAMPs

### 8.1 In Vitro Dark Cell Viability Analysis.

The in vitro biocompatibility of the nanoLAMPs with light irradiation was evaluated by testing their cytotoxicity to normal cells utilizing MTT assay. HUVECs and ARPE-19 cells were incubated in 96-well plate and received relevant treatment. Then the MTT assay was conducted after 24 h incubation.

8.2 Safety profile of nanoLAMPs in vivo.

The mice of each group received the treatment shown in Example 5.2 in laser induced CNV mouse model. At the end of the treatment period, blood of each mouse was collected into microvette capillary blood collection tubes (˜0.1 mL) by intracardiac puncture under deep anaesthesia for CBC/w test (RBC, WBC, Hb, Platelet), and microtainer serum separator tubes (˜0.8 mL) for biochemical tests. The serum of each group was isolated by centrifuge the whole blood stored in 4° C. overnight and levels of AST, ALT, UREA, CREA was detected to determine the kidney and liver function. The mice were sacrificed by cardiac perfusion with 0.9% saline followed by 4% paraformaldehyde at the end of the studies. Main organs and tumors were excised, imaged, and weighted, and the tissues were cut into 10-μm-thick cryostat slices for H&E staining to assess the in vivo biocompatibility.

### 8.3 Electroretinogram

The mice were randomly dived into two groups, the control group and treatment group. The treatment group receives nanoLAMPs intravenous injection (containing 2 mg AS16 peptide) twice with an interval of 1 day, while the control group receives intravenous injections of PBS at the same time point. After an additional day, the mice were carefully housed in dark rooms for dark adaptation overnight. Consequently, the mice were anesthetized, and the pupils were dilated with 0.5% tropicamide, followed by tetracaine eye drops for local anesthesia. fERG was recorded with gold plated wire loop electrodes contacting the corneal surface as the active electrode. The mice were exposed to full-field scotopic flashes with intensity of 0.003, 0.01, 0.03, 0.1, 0.3, 1.0, 3.0, and 10.0 log cd.s/m².

8.4 TUNEL assay

The mice received nanoLAMPs intravenous injections and euthanized one day after treatment. The eyes were harvested, and paraffin sections were conducted. The sections of nanoLAMPs plus light irradiation group and saline group were then incubated with TUNEL assay kit (Servicebio Wuhan, China) following the instructions and the images were captured by confocal microscope.

### 8.5 Results

As shown in FIG. 9a, the NIR light as well as the nanoLAMPs treatment did not exert obvious damages to cornea, as no obvious neovascularization or edema was observed. Moreover, the TUNEL assay of retina showed no obvious difference between control group and nanoLAMPs treatment group (FIG. 9b) at one day after the second treatment, indicating that our treatment did not induce cell apoptosis in the retina. The TUNEL assay of CNV lesions was served as positive control (FIG. 12). Moreover, the nanoLAMPs treatment did not decrease the thickness of inner nuclear layer (INL) and outer nuclear layer (ONL) of the retinal, which further confirmed that our treatment modality should be safe for ocular use (FIGS. 9a and 9c). More importantly, the results of flash electroretinogram (fERG), which has a high sensitivity in detecting retina damage showed no significant difference between nanoLAMPs treatment group and control group in scotopic a-wave and b-wave (FIG. 9d-9f). These results demonstrated that our nanoLAMPs treatment did not induce obvious damage to cornea and retina and should be safe for ocular applications.

Furthermore, the H&E sections of main organs of each group including heart, liver, spleen, lung, kidney were conducted to finally determine the safety profile of our nanoLAMPs treatment. As shown in FIG. 10a, no obvious morphology change was observed in all treatment groups. The whole blood sample and serum were also collected and isolated for biocompatibility investigations. As shown in FIG. 10b, no obvious changes in the RBC, WBC, platelet counts in each group were noticed and Hb concentration of each group exerted negligible difference (FIG. 10c). Moreover, the concentration of ALT, AST, UREA and CREA of each group remained the same after each treatment, indicating a well preserved hepatorenal function by our nanoLAMPs treatment. Also, the MTT assay of HUVECs and ARPE-19 cell line showed that our nanoparticles did not exert obvious cytotoxicity effect in dark conditions (FIG. 10d), indicating that our nanoparticle should be safe for systemic administration. Finally, the body weight of the mice in each group did not show significantly difference for 15 days observation (FIG. 10e). These results all demonstrated that our nanoLAMPs treatment is safe to use with no obvious systemic or local adverse events.

### Example 9. Nanoparticle Encapsulating Additional Agents

To further investigate whether the system can be used to deliver hydrophobic drugs, an anti-cancer drugs elesclomol-Cu is incoporated to prepare nanoparticles with BODIPY-caged-AS16 (cAS16) and PLA5k-PEG2k. 0.5 μL of 10 mg/mL elesclomol was added to 200 μL 0.1 mg/mL CuCl₂·2H₂O solution to prepare the elesclomol-Cu complexes. Subsequently, the cAS16&PLA5k-PEG2k DMSO mixture (4 μL 20 mg/ml cAS16+4 μL 100 mg/ml PLA5k-PEG2k) was added to the above solution under vigorous stirring to make the components self-assemble to nanoparticles. Then the nanoparticles were concentrated by centrifuge at 18000 rpm for 30 min. The aggregates were removed also by centrifuge at 4000 rpm for 5 min. The supernatant was used for characterization and cell experiments. As shown in FIG. 11a, elesclomol-Cu can be successfully encapsulated by cAS16/PLA5k-PEG2k. The nanoparticles were prepared by the nanoprecipitation method. Through ultrafiltration, free peptides and drugs were removed. The encapsulation efficiency and loading content of cAS16 were 75.34% and 20.08%, respectively. The encapsulation efficiency and loading content of elesclomol-Cu were 13.25% and 8.82%, respectively (FIG. 11b). After successful nanoparticle preparation, CT26 cells were incubated with the nanoparticles. As shown in FIG. 11c, cAS16 exhibited no cytotoxicity after light irradiation. And elesclomol-Cu-loaded nanoparticles with or without light irradiation showed similar cytotoxicity to free elesclomol-Cu. Such results indicated that the system can be used to encapsulate hydrophobic small molecules for combination therapy or synergistic therapy.

### Example 10. BODIPY-Caged FM12 Peptide for Photo-Triggered PD-L1 Blockade

To further explore whether the BODIPY photocage can be utilized to deliver other hydrophilic peptides, a peptide inhibitor (FPNWSLRPMNQM, SEQ ID NO: 2, FM12) targeting programmed death ligand 1 (PD-L1) was conjugated with BODIPY at N-terminus to synthesize BODIPY-caged FM12 (cFM12). The conjugate could also form stable polymeric nanoparticles with the help of PEG3.4K-PLA5K. As shown in FIGS. 13a and 13b, FM12 peptide could be released from cFM12 successfully upon red-light irradiation (656 nm, 30 mW/cm²) in a time-dependent manner. Besides, the released FM12 peptide could promote the gene expression level of pro-inflammatory cytokines such as IL-2 and TNF-α in Jurkat T cells after co-culture with MDA-MB-231 cells, when compared with the control group treated with the same amount of PBS (FIGS. 13c and 13d). For nanoparticle preparation, in brief, 2 μL of 10 mg/mL cFM12 was mixed with 2 μL of 100 mg/mL PEG3.4K-PLA5K in DMSO solution. Subsequently, the 4 μL cFM12 and PEG3.4K-PLA5K DMSO mixture was added to 200 μL aqueous solution under vigorous stirring to make the components self-assemble to nanoparticles. Then the nanoparticles were concentrated by centrifuge at 18000 rpm for 30 min. The aggregates were removed by centrifuge at 4000 rpm for 5 min. The supernatant was used for characterization and cell experiments. As shown in FIGS. 13e and 13f, the fabricated nanoparticles had well-dispersed size distribution with diameter around 122 nm and PDI around 0.2. Those results demonstrated that this strategy could also be applied to the other peptide for successful photocaging and nanoparticle formation for red light-triggered PD-L1 blockade and cancer immunotherapy.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

SEQUENCE LISITNG
SEQ ID NO: 1
ATWLPPRAANLLMAAS
SEQ ID NO: 2
FPNWSLRPMNQM

1. (canceled)

2. A photoresponsive prodrug-comprising a peptide conjugated to a photoresponsive group, wherein the peptide is an anti-angiogenic peptide AS16 peptide comprising an amino acid sequence as shown in SEQ ID NO: 1 (ATWLPPRAANLLMAAS) or FM12 peptide comprising an amino acid sequence as shown in SEQ ID NO: 2 (FPNWSLRPMNQM).

3. The photoresponsive prodrug of claim 2, wherein the photoresponsive group is boron-dipyrromethene (BODIPY), preferably, the BODIPY is a moiety selected from formula (VII), formula (VIII), formula (IX), formula (X), formula (XI), or formula (XII), more preferably, the BODIPY is a moiety of formula (XII):

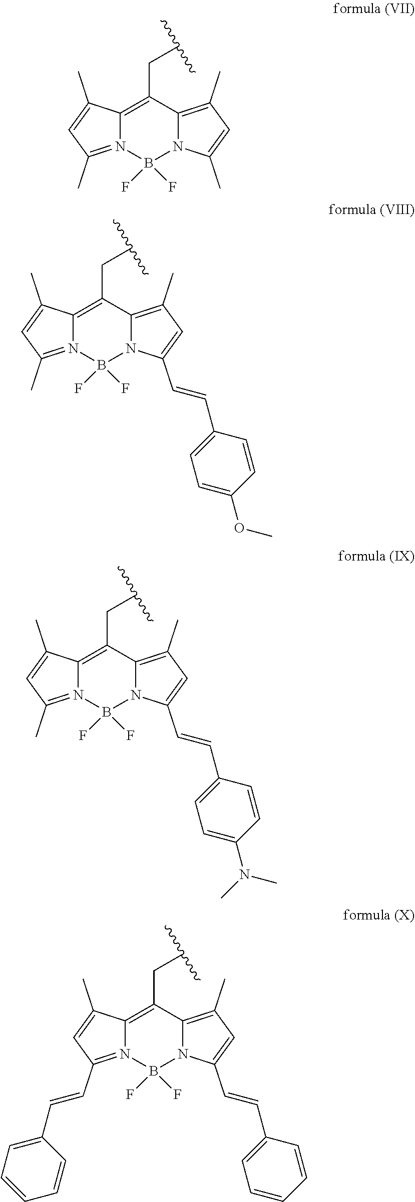

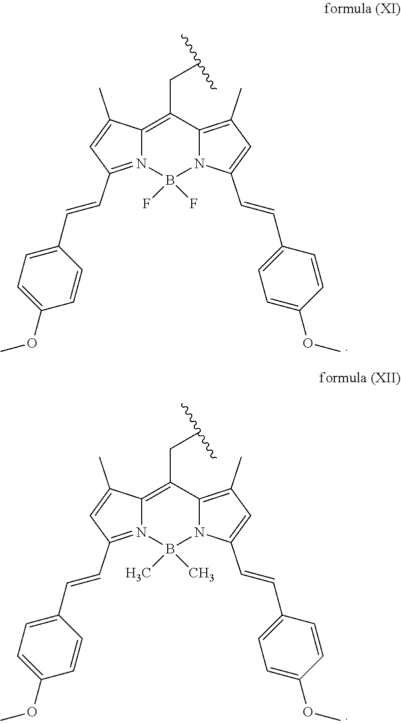

4. The photoresponsive prodrug of claim 2, wherein the photoresponsive prodrug is 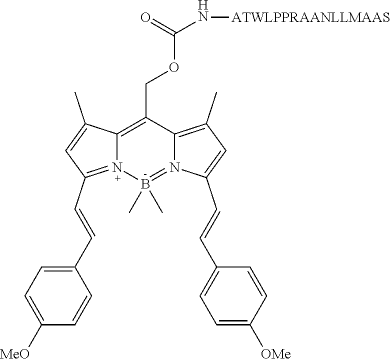
or cFM12:

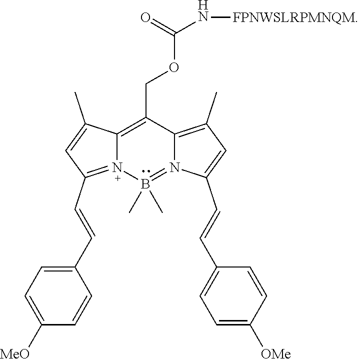

5. A nanoparticle, comprising the photoresponsive prodrug of claim 2, and one or more polymers.

6. The nanoparticle of claim 5, wherein the polymer is selected from polylactic acid (PLA), polyethylene glycol (PEG), polydimethylsiloxane (PDMS), polyethyleneimine (PEI), polyamidoamine (PAMAM), and a combination thereof; preferably, the polymer is selected from PLA-PEG copolymer; more preferably, the polymer is PLA5k-PEG2k or PEG3.4k-PLA5k.

7. The nanoparticle of claim 5, having a diameter of about 20-200 nm.

8. The nanoparticle of claim 7, having a polydispersity index (PDI) of 0.120-0.220.

9. The nanoparticle of claim 7, having a diameter of about 60 nm, and a PDI of 0.170; or having a diameter of about 122 nm, and a PDI of 0.2.

10. The nanoparticle of claim 5, comprising a photoresponsive prodrug of the following formula co-assembled with PLA5k-PEG2k 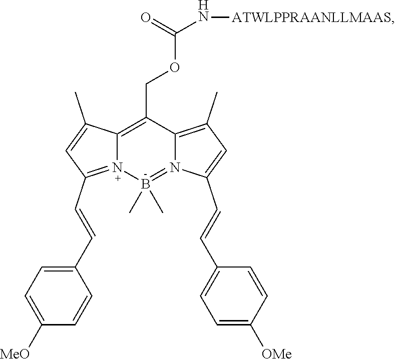
or a photoresponsive prodrug of the following formula of cFM12 co-assembled with PLA3.4k-PLA5k

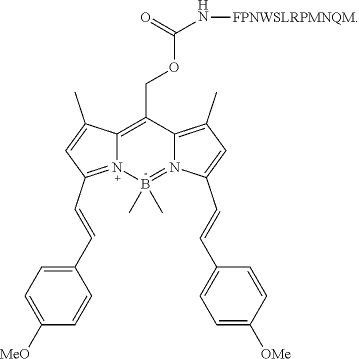

11. The nanoparticle of claim 5, further encapsulating additional agents; preferably, the additional agents are hydrophobic drugs; preferably, the additional agents are anti-cancer drugs; more preferably, the additional agents are selected from tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfm, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, or iniparib; further more preferably, the additional agent is elesclomol.

12. A pharmaceutical composition, comprising: (i) the photoresponsive prodrug of claim 2; and (ii) a pharmaceutically acceptable excipient.

13. A method of delivering a drug to a subject at a target site, comprising: (i) administering the photoresponsive prodrug of claim 2 to the subject; and (ii) irradiating the self-assembly system at the target site with a light source.

14. The method of claim 13, wherein the light source has a wavelength of about 500-1300 nm.

15. The method of claim 13- or 14, wherein the photoresponsive prodrug or the nanoparticle is administered via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, intratumoral administration, and any combination thereof; preferably via intravenous administration.

16. The method of claim 13, wherein the target site is eye, skin, or tumor, preferably posterior segment of the eye.

17. A method for treating or preventing a disease characterized by abnormal angiogenesis, comprising: (i) administering the photoresponsive prodrug of claim 2 to the subject; and (ii) irradiating the self-assembly system at the target site with a light source.

18. The method of claim 17, wherein the light source has a wavelength of about 500-1300 nm.

19. The method of claim 17, wherein the photoresponsive prodrug is administered via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, intratumoral administration, and any combination thereof; preferably via intravenous administration.

20. (canceled)

21. (canceled)

22. (canceled)

23. A kit comprising the photoresponsive prodrug of claim 2.
